SEQUENCE LISTING (296 sequences)

SEQ ID NO: 1 - DNA, 1312 bp, mouse
gcgtcgacactagttctagaccctcattggaccctcgtacagaagctaatacgactcactatagggaaataagagagaaaagaagagtaagaagaaatataagagccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgagctgccttctgcggggcttgccttctggccatgcccttcttctctcccttgcacctgtacctcttggtctttgaataaagcctgagtaggaagtgagggtctagaactagtgtcgacgc

SEQ ID NO: 2 - protein, 360 aa, mouse
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - DNA, 1183 bp, Homo sapiens
gcgtcgacactagttctagaccctcattggaccctcgtacagaagctaatacgactcactatagggaaataagagagaaaagaagagtaagaagaaatataagagccaccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagctgccttctgcggggcttgccttctggccatgcccttcttctctcccttgcacctgtacctcttggtctttgaataaagcctgagtaggaagtgagggtctagaactagtgtcgacgc

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 6 - DNA, 859 bp, Homo sapiens
gcgtcgacactagttctagaccctcattggaccctcgtacagaagctaatacgactcactatagggaaataagagagaaaagaagagtaagaagaaatataagagccaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgagctgccttctgcggggcttgccttctggccatgcccttcttctctcccttgcacctgtacctcttggtctttgaataaagcctgagtaggaagtgagggtctagaactagtgtcgacgc

SEQ ID NO: 7 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 8 - DNA, 1642 bp, Homo sapiens
gcgtcgacactagttctagaccctcattggaccctcgtacagaagctaatacgactcactatagggaaataagagagaaaagaagagtaagaagaaatataagagccaccatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaagctgccttctgcggggcttgccttctggccatgcccttcttctctcccttgcacctgtacctcttggtctttgaataaagcctgagtaggaagtgagggtctagaactagtgtcgacgc

SEQ ID NO: 9 - protein, 504 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 10 - DNA, 1642 bp, Homo sapiens; identical to SEQ ID NO: 8

SEQ ID NO: 11 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 12 - DNA, 1051 bp, Homo sapiens
gcgtcgacactagttctagaccctcattggaccctcgtacagaagctaatacgactcactatagggaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagagatctcgagctgatccaaaaaagaagagaaaggtagatccaaaaaagaagagaaaggtagatccaaaaaagaagagaaaggtaggatccaccggatctagataagctgccttctgcggggcttgccttctggccatgcccttcttctctcccttgcacctgtacctcttggtctttgaataaagcctgagtaggaagtgagggtctagaactagtgtcgacgc

SEQ ID NO: 13 - DNA, 69 bp, artificial
  hsa-mir-302a
ccaccacuuaaacguggauguacuugcuuugaaacuaaagaaguaagugcuuccauguuuuggugaugg

SEQ ID NO: 14 - DNA, 23 bp, artificial
  hsa-mir-302a
uaagugcuuccauguuuugguga

SEQ ID NO: 15 - DNA, 73 bp, artificial
  hsa-mir-302b
gcucccuucaacuuuaacauggaagugcuuucugugacuuuaaaaguaagugcuuccauguuuuaguaggagu

SEQ ID NO: 16 - DNA, 23 bp, artificial
  hsa-mir-302b
uaagugcuuccauguuuuaguag

SEQ ID NO: 17 - DNA, 68 bp, artificial
  hsa-mir-302c
ccuuugcuuuaacauggggguaccugcugugugaaacaaaaguaagugcuuccauguuucaguggagg

SEQ ID NO: 18 - DNA, 23 bp, artificial
  hsa-mir-302c
uaagugcuuccauguuucagugg

SEQ ID NO: 19 - DNA, 68 bp, artificial
  hsa-mir-302d
ccucuacuuuaacauggaggcacuugcugugacaugacaaaaauaagugcuuccauguuugagugugg

SEQ ID NO: 20 - DNA, 23 bp, artificial
  hsa-mir-302d
uaagugcuuccauguuugagugu

SEQ ID NO: 21 - DNA, 68 bp, artificial
  hsa-mir-367
ccauuacuguugcuaauaugcaacucuguugaauauaaauuggaauugcacuuuagcaauggugaugg

SEQ ID NO: 22 - DNA, 22 bp, artificial
  hsa-mir-367
aauugcacuuuagcaaugguga

SEQ ID NO: 23 - DNA, 68 bp, artificial
  hsa-mir-200c
cccucgucuuacccagcaguguuugggugcgguugggagucucuaauacugccggguaaugauggagg

SEQ ID NO: 24 - DNA, 21 bp, artificial
  hsa-mir-369-3p
aauaauacaugguugaucuuu

SEQ ID NO: 25 - DNA, 22 bp, artificial
  hsa-mir-369-5p
agaucgaccguguuauauucgc

SEQ ID NO: 26 - DNA, 23 bp, artificial
  mmu-miR-302b
uaagugcuuccauguuuuaguag

SEQ ID NO: 27 - DNA, 23 bp, artificial
  mmu-miR-302
uaagugcuuccauguuuugguga

SEQ ID NO: 28 - DNA, 22 bp, artificial
  mmu-miR-495
aaacaaacauggugcacuucuu

SEQ ID NO: 29 - DNA, 22 bp, artificial
  mmu-miR-26a
uucaaguaauccaggauaggcu

SEQ ID NO: 30 - DNA, 22 bp, artificial
  mmu-miR-19a
uaguuuugcauaguugcacuac

SEQ ID NO: 31 - DNA, 23 bp, artificial
  mmu-miR-302d
uaagugcuuccauguuugagugu

SEQ ID NO: 32 - DNA, 23 bp, artificial
  mmu-miR-10b
uacccuguagaaccgaauuugug

SEQ ID NO: 33 - DNA, 22 bp, artificial
  mmu-miR-294
aaagugcuucccuuuugugugu

SEQ ID NO: 34 - DNA, 22 bp, artificial
  mmu-miR-302c
aagugcuuccauguuucagugg

SEQ ID NO: 35 - DNA, 22 bp, artificial
  mmu-miR-183
gugaauuaccgaagggccauaa

SEQ ID NO: 36 - DNA, 22 bp, artificial
  mmu-miR-200a
uaacacugucugguaacgaugu

SEQ ID NO: 37 - DNA, 22 bp, artificial
  mmu-miR34c
aaucacuaaccacacagccagg

SEQ ID NO: 38 - DNA, 22 bp, artificial
  mmu-miR-293
agugccgcagaguuuguagugu

SEQ ID NO: 39 - DNA, 23 bp, artificial
  mmu-miR-181b
aacauucauugcugucggugggu

SEQ ID NO: 40 - DNA, 21 bp, artificial
  mmu-miR-151
cuagacugaggcuccuugagg

SEQ ID NO: 41 - DNA, 21 bp, artificial
  mmu-miR-680
gggcaucugcugacauggggg

SEQ ID NO: 42 - DNA, 23 bp, artificial
  mmu-miR-295
aaagugcuacuacuuuugagucu

SEQ ID NO: 43 - DNA, 22 bp, artificial
  mmu-miR-880
uacuccauccucucugaguaga

SEQ ID NO: 44 - DNA, 23 bp, artificial
  mmu-miR-93
caaagugcuguucgugcagguag

SEQ ID NO: 45 - DNA, 22 bp, artificial
  mmu-miR-455-5p
uaugugccuuuggacuacaucg

SEQ ID NO: 46 - DNA, 20 bp, artificial
  mmu-miR-144
uacaguauagaugauguacu

SEQ ID NO: 47 - DNA, 22 bp, artificial
  mmu-miR-467d
uaagugcgcgcauguauaugcg

SEQ ID NO: 48 - DNA, 22 bp, artificial
  mmu-miR-484
ucaggcucaguccccucccgau

SEQ ID NO: 49 - DNA, 22 bp, artificial
  mmu-miR-205
uccuucauuccaccggagucug

SEQ ID NO: 50 - DNA, 22 bp, artificial
  mmu-miR-582-5p
uacaguuguucaaccaguuacu

SEQ ID NO: 51 - DNA, 24 bp, artificial
  mmu-miR-290-3p
aaagugccgccuaguuuuaagccc

SEQ ID NO: 52 - DNA, 23 bp, artificial
  mmu-miR-290-3p
aaagugccgccuaguuuuaagcc

SEQ ID NO: 53 - DNA, 22 bp, artificial
  mmu-miR-138
cggcuacuucacaacaccaggg

SEQ ID NO: 54 - DNA, 23 bp, artificial
  mmu-miR-181d
aacauucauuguugucggugggu

SEQ ID NO: 55 - DNA, 20 bp, artificial
  mmu-miR-324-3p
ccacugccccaggugcugcu

SEQ ID NO: 56 - DNA, 22 bp, artificial
  mmu-miR-877
uguccucuucucccuccuccca

SEQ ID NO: 57 - DNA, 21 bp, artificial
  mmu-miR-23a
aucacauugccagggauuucc

SEQ ID NO: 58 - DNA, 21 bp, artificial
  mmu-miR-379
ugguagacuauggaacguagg

SEQ ID NO: 59 - DNA, 22 bp, artificial
  mmu-miR-673
cucacagcucugguccuuggag

SEQ ID NO: 60 - DNA, 22 bp, artificial
  mmu-miR-876-5p
uggauuucucugugaaucacua

SEQ ID NO: 61 - DNA, 22 bp, artificial
  mmu-miR-291-3p
aaagugcuuccacuuugugugc

SEQ ID NO: 62 - DNA, 22 bp, artificial
  mmu-miR-30d
uguaaacauccccgacuggaag

SEQ ID NO: 63 - DNA, 23 bp, artificial
  mmu-miR-421
aucaacagacauuaauugggcgc

SEQ ID NO: 64 - DNA, 22 bp, artificial
  mmu-miR-879
gcuuauggcuucaagcuuucgg

SEQ ID NO: 65 - DNA, 22 bp, artificial
  mmu-miR-542-3p
ugugacagauugauaacugaaa

SEQ ID NO: 66 - DNA, 22 bp, artificial
  mmu-miR-124
cguguucacagcggaccuugau

SEQ ID NO: 67 - DNA, 22 bp, artificial
  mmu-miR-363
aauugcacgguauccaucugua

SEQ ID NO: 68 - DNA, 23 bp, artificial
  mmu-miR-871
uauucagauuagugccagucaug

SEQ ID NO: 69 - DNA, 23 bp, artificial
  mmu-miR-19a
ugugcaaaucuaugcaaaacuga

SEQ ID NO: 70 - DNA, 22 bp, artificial
  mmu-miR-16
ccaguauugacugugcugcuga

SEQ ID NO: 71 - DNA, 21 bp, artificial
  mmu-miR-873
gcaggaacuugugagucuccu

SEQ ID NO: 72 - DNA, 23 bp, artificial
  mmu-miR-199b
cccaguguuuagacuaccuguuc

SEQ ID NO: 73 - DNA, 23 bp, artificial
  mmu-miR-106a
caaagugcuaacagugcagguag

SEQ ID NO: 74 - DNA, 23 bp, artificial
  mmu-miR-181b
aacauucauugcugucggugggu

SEQ ID NO: 75 - DNA, 22 bp, artificial
  mmu-miR-200a
caucuuaccggacagugcugga

SEQ ID NO: 76 - DNA, 22 bp, artificial
  mmu-miR-431
caggucgucuugcagggcuucu

SEQ ID NO: 77 - DNA, 21 bp, artificial
  mmu-miR-689
cguccccgcucggcggggucc

SEQ ID NO: 78 - DNA, 21 bp, artificial
  mmu-miR-721
cagugcaauuaaaagggggaa

SEQ ID NO: 79 - DNA, 22 bp, artificial
  mmu-miR-744
cuguugccacuaaccucaaccu

SEQ ID NO: 80 - DNA, 23 bp, artificial
  mmu-miR-423-5p
ugaggggcagagagcgagacuuu

SEQ ID NO: 81 - DNA, 22 bp, artificial
  mmu-miR-669c
auaguuguguguggaugugugu

SEQ ID NO: 82 - DNA, 22 bp, artificial
  mmu-let-7c
ugagguaguagguuguaugguu

SEQ ID NO: 83 - DNA, 22 bp, artificial
  mmu-miR-466h
ugugugcaugugcuugugugua

SEQ ID NO: 84 - DNA, 22 bp, artificial
  mmu-miR-654-3p
uaugucugcugaccaucaccuu

SEQ ID NO: 85 - DNA, 22 bp, artificial
  mmu-miR-470
aaccaguaccuuucugagaaga

SEQ ID NO: 86 - DNA, 22 bp, artificial
  mmu-miR-24
uggcucaguucagcaggaacag

SEQ ID NO: 87 - DNA, 25 bp, artificial
  mmu-miR-182
uuuggcaaugguagaacucacaccg

SEQ ID NO: 88 - DNA, 23 bp, artificial
  mmu-miR-335
ucaagagcaauaacgaaaaaugu

SEQ ID NO: 89 - DNA, 22 bp, artificial
  mmu-miR-181c
aacauucaaccugucggugagu

SEQ ID NO: 90 - DNA, 23 bp, artificial
  mmu-miR-330
gcaaagcacagggccugcagaga

SEQ ID NO: 91 - DNA, 22 bp, artificial
  mmu-miR-134
ugugacugguugaccagagggg

SEQ ID NO: 92 - DNA, 22 bp, artificial
  mmu-miR-675-3p
cuguaugcccuaaccgcucagu

SEQ ID NO: 93 - DNA, 21 bp, artificial
  mmu-miR-218
uugugcuugaucuaaccaugu

SEQ ID NO: 94 - DNA, 22 bp, artificial
  mmu-let-7f
ugagguaguagguuguaugguu

SEQ ID NO: 95 - DNA, 22 bp, artificial
  mmu-miR-491
aguggggaacccuuccaugagg

SEQ ID NO: 96 - DNA, 21 bp, artificial
  mmu-miR-466g
auacagacacaugcacacaca

SEQ ID NO: 97 - DNA, 22 bp, artificial
  mmu-miR-465c-3p
gaucagggccuuucuaaguaga

SEQ ID NO: 98 - DNA, 22 bp, artificial
  mmu-miR-202
agagguauagcgcaugggaaga

SEQ ID NO: 99 - DNA, 21 bp, artificial
  mmu-miR-681
cagccucgcuggcaggcagcu

SEQ ID NO: 100 - DNA, 20 bp, artificial
  mmu-miR-877
guagaggagauggcgcaggg

SEQ ID NO: 101 - DNA, 22 bp, artificial
  mmu-miR-875-5p
uauaccucaguuuuaucaggug

SEQ ID NO: 102 - DNA, 21 bp, artificial
  mmu-miR-712
cuccuucacccgggcgguacc

SEQ ID NO: 103 - DNA, 22 bp, artificial
  mmu-miR-297
auguaugugugcaugugcaugu

SEQ ID NO: 104 - DNA, 22 bp, artificial
  mmu-let-7d
agagguaguagguugcauaguu

SEQ ID NO: 105 - DNA, 23 bp, artificial
  mmu-miR-142-3p
uguaguguuuccuacuuuaugga

SEQ ID NO: 106 - DNA, 22 bp, artificial
  mmu-miR-328
cuggcccucucugcccuuccgu

SEQ ID NO: 107 - DNA, 22 bp, artificial
  mmu-miR-485-5p
agaggcuggccgugaugaauuc

SEQ ID NO: 108 - DNA, 22 bp, artificial
  mmu-miR-122a
uggagugugacaaugguguuug

SEQ ID NO: 109 - DNA, 22 bp, artificial
  mmu-miR-877
uguccucuucucccuccuccca

SEQ ID NO: 110 - DNA, 23 bp, artificial
  mmu-miR-135a
uauggcuuuuuauuccuauguga

SEQ ID NO: 111 - DNA, 22 bp, artificial
  mmu-miR-674-3p
cacagcucccaucucagaacaa

SEQ ID NO: 112 - DNA, 22 bp, artificial
  mmu-miR-497
cagcagcacacugugguuugua

SEQ ID NO: 113 - DNA, 23 bp, artificial
  mmu-miR-7b
uggaagacuugugauuuuguugu

SEQ ID NO: 114 - DNA, 22 bp, artificial
  mmu-miR-30b
cugggauguggauguuuacguc

SEQ ID NO: 115 - DNA, 23 bp, artificial
  mmu-miR-34b
aggcaguguaauuagcugauugu

SEQ ID NO: 116 - DNA, 22 bp, artificial
  mmu-miR-466e-5p
gauguguguguacauguacaua

SEQ ID NO: 117 - DNA, 22 bp, artificial
  mmu-miR-193b
aacuggcccacaaagucccgcu

SEQ ID NO: 118 - DNA, 22 bp, artificial
  mmu-miR-883a-5p
ugcugagagaaguagcaguuac

SEQ ID NO: 119 - DNA, 22 bp, artificial
  mmu-let-7i
cugcgcaagcuacugccuugcu

SEQ ID NO: 120 - DNA, 23 bp, artificial
  mmu-miR-342
ucucacacagaaaucgcacccgu

SEQ ID NO: 121 - DNA, 21 bp, artificial
  mmu-miR-140
uaccacaggguagaaccacgg

SEQ ID NO: 122 - DNA, 22 bp, artificial
  mmu-miR-24-2
gugccuacugagcugaaacagu

SEQ ID NO: 123 - DNA, 21 bp, artificial
  mmu-miR-195
uagcagcacagaaauauuggc

SEQ ID NO: 124 - DNA, 22 bp, artificial
  mmu-miR-297a
auguaugugugcaugugcaugu

SEQ ID NO: 125 - DNA, 23 bp, artificial
  mmu-miR-344
ugaucuagccaaagccugacugu

SEQ ID NO: 126 - DNA, 23 bp, artificial
  mmu-miR-18
uaaggugcaucuagugcagauag

SEQ ID NO: 127 - DNA, 22 bp, artificial
  mmu-miR-93
acugcugagcuagcacuucccg

SEQ ID NO: 128 - DNA, 22 bp, artificial
  mmu-miR-297
auguaugugugcaugugcaugu

SEQ ID NO: 129 - DNA, 22 bp, artificial
  mmu-miR-16
uagcagcacguaaauauuggcg

SEQ ID NO: 130 - DNA, 22 bp, artificial
  mmu-miR-380-5p
augguugaccauagaacaugcg

SEQ ID NO: 131 - DNA, 23 bp, artificial
  mmu-miR-672
ugagguugguguacuguguguga

SEQ ID NO: 132 - DNA, 21 bp, artificial
  mmu-miR-431
ugucuugcaggccgucaugca

SEQ ID NO: 133 - DNA, 21 bp, artificial
  mmu-miR-715
cuccgugcacacccccgcgug

SEQ ID NO: 134 - DNA, 22 bp, artificial
  mmu-miR-669a
aguugugugugcauguucaugu

SEQ ID NO: 135 - DNA, 23 bp, artificial
  mmu-miR-103
agcagcauuguacagggcuauga

SEQ ID NO: 136 - DNA, 22 bp, artificial
  mmu-miR-124
cguguucacagcggaccuugau

SEQ ID NO: 137 - DNA, 22 bp, artificial
  mmu-miR-15b
uagcagcacaucaugguuuaca

SEQ ID NO: 138 - DNA, 22 bp, artificial
  mmu-miR-450b
auugggaacauuuugcaugcau

SEQ ID NO: 139 - DNA, 22 bp, artificial
  mmu-miR-882
aggagagaguuagcgcauuagu

SEQ ID NO: 140 - DNA, 22 bp, artificial
  mmu-miR-686
auugcuucccagacggugaaga

SEQ ID NO: 141 - DNA, 21 bp, artificial
  mmu-miR-222
agcuacaucuggcuacugggu

SEQ ID NO: 142 - DNA, 19 bp, artificial
  mmu-miR-684
aguuuucccuucaagucaa

SEQ ID NO: 143 - DNA, 22 bp, artificial
  mmu-miR-450b
uuuugcaguauguuccugaaua

SEQ ID NO: 144 - DNA, 22 bp, artificial
  mmu-miR-582-3p
ccuguugaacaacugaacccaa

SEQ ID NO: 145 - DNA, 23 bp, artificial
  mmu-miR-135b
uauggcuuuucauuccuauguga

SEQ ID NO: 146 - DNA, 23 bp, artificial
  mmu-miR-493
ugaagguccuacugugugccagg

SEQ ID NO: 147 - DNA, 16 bp, artificial
  mmu-miR-546
augguggcacggaguc

SEQ ID NO: 148 - DNA, 23 bp, artificial
  mmu-miR-708
aaggagcuuacaaucuagcuggg

SEQ ID NO: 149 - DNA, 22 bp, artificial
  mmu-miR-433-3p
aucaugaugggcuccucggugu

SEQ ID NO: 150 - DNA, 22 bp, artificial
  mmu-miR-494
ugaaacauacacgggaaaccuc

SEQ ID NO: 151 - DNA, 22 bp, artificial
  mmu-miR-203
gugaaauguuuaggaccacuag

SEQ ID NO: 152 - DNA, 23 bp, artificial
  mmu-miR-9
ucuuugguuaucuagcuguauga

SEQ ID NO: 153 - DNA, 23 bp, artificial
  mmu-miR-574-5p
ugagugugugugugugagugugu

SEQ ID NO: 154 - DNA, 21 bp, artificial
  mmu-miR-376c
aacauagaggaaauuucacgu

SEQ ID NO: 155 - DNA, 22 bp, artificial
  mmu-miR-433-5p
uacggugagccugucauuauuc

SEQ ID NO: 156 - DNA, 22 bp, artificial
  mmu-miR-181a-2
accgaccguugacuguaccuug

SEQ ID NO: 157 - DNA, 22 bp, artificial
  mmu-miR-218-2
caugguucugucaagcaccgcg

SEQ ID NO: 158 - DNA, 22 bp, artificial
  mmu-miR-196a
uagguaguuucauguuguuggg

SEQ ID NO: 159 - DNA, 22 bp, artificial
  mmu-miR-542-5p
cucggggaucaucaugucacga

SEQ ID NO: 160 - DNA, 23 bp, artificial
  mmu-miR-7
uggaagacuagugauuuuguugu

SEQ ID NO: 161 - DNA, 21 bp, artificial
  mmu-miR-743b-5p
uguucagacugguguccauca

SEQ ID NO: 162 - DNA, 22 bp, artificial
  mmu-miR-377
aucacacaaaggcaacuuuugu

SEQ ID NO: 163 - DNA, 21 bp, artificial
  mmu-miR-683
ccugcuguaagcuguguccuc

SEQ ID NO: 164 - DNA, 22 bp, artificial
  mmu-miR-675-5p
uggugcggaaagggcccacagu

SEQ ID NO: 165 - DNA, 22 bp, artificial
  mmu-miR-598
uacgucaucgucgucaucguua

SEQ ID NO: 166 - DNA, 22 bp, artificial
  mmu-miR15b
cgaaucauuauuugcugcucua

SEQ ID NO: 167 - DNA, 23 bp, artificial
  mmu-miR-9
ucuuugguuaucuagcuguauga

SEQ ID NO: 168 - DNA, 22 bp, artificial
  mmu-miR-450a-3p
auuggggaugcuuugcauucau

SEQ ID NO: 169 - DNA, 20 bp, artificial
  mmu-miR-449b
aggcaguguuguuagcuggc

SEQ ID NO: 170 - DNA, 21 bp, artificial
  mmu-miR-707
cagucaugccgcuugccuacg

SEQ ID NO: 171 - DNA, 22 bp, artificial
  mmu-miR-335-3p
uuuuucauuauugcuccugacc

SEQ ID NO: 172 - DNA, 22 bp, artificial
  mmu-miR-147
gugugcggaaaugcuucugcua

SEQ ID NO: 173 - DNA, 22 bp, artificial
  mmu-miR-466c-5p
gaugugugugugcauguacaua

SEQ ID NO: 174 - DNA, 22 bp, artificial
  mmu-miR-16
uagcagcacguaaauauuggcg

SEQ ID NO: 175 - DNA, 22 bp, artificial
  mmu-miR-127
ucggauccgucugagcuuggcu

SEQ ID NO: 176 - DNA, 23 bp, artificial
  mmu-miR- 673-3p
uccggggcugaguucugugcacc

SEQ ID NO: 177 - DNA, 22 bp, artificial
  mmu-miR- 466b-5p
gauguguguguacauguacaug

SEQ ID NO: 178 - DNA, 22 bp, artificial
  mmu-miR- 27a
agggcuuagcugcuugugagca

SEQ ID NO: 179 - DNA, 22 bp, artificial
  mmu-miR-1
uggaauguaaagaaguauguau

SEQ ID NO: 180 - DNA, 22 bp, artificial
  mmu-miR-201
uacucaguaaggcauuguucuu

SEQ ID NO: 181 - DNA, 21 bp, artificial
  mmu-miR-376b
aucauagaggaacauccacuu

SEQ ID NO: 182 - DNA, 22 bp, artificial
  mmu-miR-187
ucgugucuuguguugcagccgg

SEQ ID NO: 183 - DNA, 22 bp, artificial
  mmu-miR-299
ugguuuaccgucccacauacau

SEQ ID NO: 184 - DNA, 22 bp, artificial
  mmu-miR-299
uaugugggacgguaaaccgcuu

SEQ ID NO: 185 - DNA, 22 bp, artificial
  mmu-miR-574-3p
cacgcucaugcacacacccaca

SEQ ID NO: 186 - DNA, 22 bp, artificial
  mmu-miR- 193
ugggucuuugcgggcaagauga

SEQ ID NO: 187 - DNA, 22 bp, artificial
  mmu-miR-679
ggacugugaggugacucuuggu

SEQ ID NO: 188 - DNA, 23 bp, artificial
  mmu-miR- 540-5p
caagggucacccucugacucugu

SEQ ID NO: 189 - DNA, 22 bp, artificial
  mmu-miR- 466a-5p
uauguguguguacauguacaua

SEQ ID NO: 190 - DNA, 23 bp, artificial
  mmu-miR-470
uucuuggacuggcacuggugagu

SEQ ID NO: 191 - DNA, 21 bp, artificial
  mmu-miR-1224
gugaggacuggggagguggag

SEQ ID NO: 192 - DNA, 23 bp, artificial
  mmu-miR-191
caacggaaucccaaaagcagcug

SEQ ID NO: 193 - DNA, 23 bp, artificial
  hsa-miR-199b-5p
cccaguguuuagacuaucuguuc

SEQ ID NO: 194 - DNA, 22 bp, artificial
  hsa-let-7a
ugagguaguagguuguauaguu

SEQ ID NO: 195 - DNA, 22 bp, artificial
  hsa-let-7b
ugagguaguagguugugugguu

SEQ ID NO: 196 - DNA, 22 bp, artificial
  hsa-let-7c
ugagguaguagguuguaugguu

SEQ ID NO: 197 - DNA, 22 bp, artificial
  hsa-let-7d
agagguaguagguugcauaguu

SEQ ID NO: 198 - DNA, 22 bp, artificial
  hsa-let-7e
ugagguaggagguuguauaguu

SEQ ID NO: 199 - DNA, 22 bp, artificial
  hsa-let-7f
ugagguaguagauuguauaguu

SEQ ID NO: 200 - DNA, 22 bp, artificial
  hsa-let-7g
ugagguaguaguuuguacaguu

SEQ ID NO: 201 - DNA, 22 bp, artificial
  hsa-let-7i
ugagguaguaguuugugcuguu

SEQ ID NO: 202 - DNA, 22 bp, artificial
  hsa-miR-100
aacccguagauccgaacuugug

SEQ ID NO: 203 - DNA, 22 bp, artificial
  hsa-miR-100
aacccguagauccgaacuugug

SEQ ID NO: 204 - DNA, 22 bp, artificial
  hsa-miR-122
uggagugugacaaugguguuug

SEQ ID NO: 205 - DNA, 22 bp, artificial
  hsa-miR-127-3p
ucggauccgucugagcuuggcu

SEQ ID NO: 206 - DNA, 21 bp, artificial
  hsa-miR-128
ucacagugaaccggucucuuu

SEQ ID NO: 207 - DNA, 21 bp, artificial
  hsa-miR-129-5p
cuuuuugcggucugggcuugc

SEQ ID NO: 208 - DNA, 22 bp, artificial
  hsa-miR-134
ugugacugguugaccagagggg

SEQ ID NO: 209 - DNA, 22 bp, artificial
  hsa-miR-140-5p
cagugguuuuacccuaugguag

SEQ ID NO: 210 - DNA, 23 bp, artificial
  hsa-miR-145
guccaguuuucccaggaaucccu

SEQ ID NO: 211 - DNA, 23 bp, artificial
  hsa-miR-149
ucuggcuccgugucuucacuccc

SEQ ID NO: 212 - DNA, 23 bp, artificial
  hsa-miR-18a
uaaggugcaucuagugcagauag

SEQ ID NO: 213 - DNA, 23 bp, artificial
  hsa-miR-18b
uaaggugcaucuagugcaguuag

SEQ ID NO: 214 - DNA, 22 bp, artificial
  hsa-miR-18b
uaaggugcaucuagugcuguua

SEQ ID NO: 215 - DNA, 22 bp, artificial
  hsa-miR-193a-3p
aacuggccuacaaagucccagu

SEQ ID NO: 216 - DNA, 23 bp, artificial
  hsa-miR-199a-5p
cccaguguucagacuaccuguuc

SEQ ID NO: 217 - DNA, 22 bp, artificial
  hsa-miR-216a
uaaucucagcuggcaacuguga

SEQ ID NO: 218 - DNA, 22 bp, artificial
  hsa-miR-216b
aaaucucugcaggcaaauguga

SEQ ID NO: 219 - DNA, 21 bp, artificial
  hsa-miR-218
uugugcuugaucuaaccaugu

SEQ ID NO: 220 - DNA, 22 bp, artificial
  hsa-miR-26a
uucaaguaauccaggauaggcu

SEQ ID NO: 221 - DNA, 21 bp, artificial
  hsa-miR-31
aggcaagaugcuggcauagcu

SEQ ID NO: 222 - DNA, 22 bp, artificial
  hsa-miR-345
gcugacuccuaguccagggcuc

SEQ ID NO: 223 - DNA, 23 bp, artificial
  hsa-miR-34c-5p
aggcaguguaguuagcugauugc

SEQ ID NO: 224 - DNA, 24 bp, artificial
  hsa-miR-362-5p
aauccuuggaaccuaggugugagu

SEQ ID NO: 225 - DNA, 21 bp, artificial
  hsa-miR-378
acuggacuuggagucagaagg

SEQ ID NO: 226 - DNA, 20 bp, artificial
  hsa-miR-384
auuccuagaaauuguucaua

SEQ ID NO: 227 - DNA, 22 bp, artificial
  hsa-miR-409-3p
gaauguugcucggugaaccccu

SEQ ID NO: 228 - DNA, 22 bp, artificial
  hsa-miR-450a
uuuugcgauguguuccuaauau

SEQ ID NO: 229 - DNA, 22 bp, artificial
  hsa-miR-450b-5p
uuuugcaauauguuccugaaua

SEQ ID NO: 230 - DNA, 22 bp, artificial
  hsa-miR-452
aacuguuugcagaggaaacuga

SEQ ID NO: 231 - DNA, 22 bp, artificial
  hsa-miR-98
ugagguaguaaguuguauuguu

SEQ ID NO: 232 - DNA, 22 bp, artificial
  hsa-miR-99a
aacccguagauccgaucuugug

SEQ ID NO: 233 - DNA, 22 bp, artificial
  hsa-miR-99b
cacccguagaaccgaccuugcg

SEQ ID NO: 234 - DNA, 22 bp, artificial
  mmu-let-7a
ugagguaguagguuguauaguu

SEQ ID NO: 235 - DNA, 22 bp, artificial
  mmu-let-7b
ugagguaguagguugugugguu

SEQ ID NO: 236 - DNA, 22 bp, artificial
  mmu-let-7c
ugagguaguagguuguaugguu

SEQ ID NO: 237 - DNA, 22 bp, artificial
  mmu-let-7d
agagguaguagguugcauaguu

SEQ ID NO: 238 - DNA, 22 bp, artificial
  mmu-let-7e
ugagguaggagguuguauaguu

SEQ ID NO: 239 - DNA, 22 bp, artificial
  mmu-let-7f
ugagguaguagauuguauaguu

SEQ ID NO: 240 - DNA, 22 bp, artificial
  mmu-let-7g
ugagguaguaguuuguacaguu

SEQ ID NO: 241 - DNA, 22 bp, artificial
  mmu-let-7i
ugagguaguaguuugugcuguu

SEQ ID NO: 242 - DNA, 22 bp, artificial
  mmu-miR-100
aacccguagauccgaacuugug

SEQ ID NO: 243 - DNA, 22 bp, artificial
  mmu-miR-100
aacccguagauccgaacuugug

SEQ ID NO: 244 - DNA, 22 bp, artificial
  mmu-miR-122
uggagugugacaaugguguuug

SEQ ID NO: 245 - DNA, 22 bp, artificial
  mmu-miR-127
ucggauccgucugagcuuggcu

SEQ ID NO: 246 - DNA, 21 bp, artificial
  mmu-miR-128
ucacagugaaccggucucuuu

SEQ ID NO: 247 - DNA, 21 bp, artificial
  mmu-miR-129-5p
cuuuuugcggucugggcuugc

SEQ ID NO: 248 - DNA, 22 bp, artificial
  mmu-miR-134
ugugacugguugaccagagggg

SEQ ID NO: 249 - DNA, 22 bp, artificial
  mmu-miR-140
cagugguuuuacccuaugguag

SEQ ID NO: 250 - DNA, 23 bp, artificial
  mmu-miR-145
guccaguuuucccaggaaucccu

SEQ ID NO: 251 - DNA, 23 bp, artificial
  mmu-miR-149
ucuggcuccgugucuucacuccc

SEQ ID NO: 252 - DNA, 23 bp, artificial
  mmu-miR-18a
uaaggugcaucuagugcagauag

SEQ ID NO: 253 - DNA, 23 bp, artificial
  mmu-miR-18b
uaaggugcaucuagugcuguuag

SEQ ID NO: 254 - DNA, 22 bp, artificial
  mmu-miR-193
aacuggccuacaaagucccagu

SEQ ID NO: 255 - DNA, 23 bp, artificial
  mmu-miR-199a-5p
cccaguguucagacuaccuguuc

SEQ ID NO: 256 - DNA, 22 bp, artificial
  mmu-miR-199b
acaguagucugcacauugguua

SEQ ID NO: 257 - DNA, 22 bp, artificial
  mmu-miR-216a
uaaucucagcuggcaacuguga

SEQ ID NO: 258 - DNA, 22 bp, artificial
  mmu-miR-216b
aaaucucugcaggcaaauguga

SEQ ID NO: 259 - DNA, 21 bp, artificial
  mmu-miR-218
uugugcuugaucuaaccaugu

SEQ ID NO: 260 - DNA, 22 bp, artificial
  mmu-miR-26a
uucaaguaauccaggauaggcu

SEQ ID NO: 261 - DNA, 22 bp, artificial
  mmu-miR-31
aggcaagaugcuggcauagcug

SEQ ID NO: 262 - DNA, 22 bp, artificial
  mmu-miR-345
gcugaccccuaguccagugcuu

SEQ ID NO: 263 - DNA, 23 bp, artificial
  mmu-miR-34c
aggcaguguaguuagcugauugc

SEQ ID NO: 264 - DNA, 24 bp, artificial
  mmu-miR-362-5p
aauccuuggaaccuaggugugaau

SEQ ID NO: 265 - DNA, 21 bp, artificial
  mmu-miR-378 (old mmu-miR-422b)
acuggacuuggagucagaagg

SEQ ID NO: 266 - DNA, 22 bp, artificial
  mmu-miR-384-3p
auuccuagaaauuguucacaau

SEQ ID NO: 267 - DNA, 22 bp, artificial
  mmu-miR-409-3p
gaauguugcucggugaaccccu

SEQ ID NO: 268 - DNA, 22 bp, artificial
  mmu-miR-450a-5p
uuuugcgauguguuccuaauau

SEQ ID NO: 269 - DNA, 22 bp, artificial
  mmu-miR-450b-5p
uuuugcaguauguuccugaaua

SEQ ID NO: 270 - DNA, 22 bp, artificial
  mmu-miR-452
uguuugcagaggaaacugagac

SEQ ID NO: 271 - DNA, 23 bp, artificial
  mmu-miR-464
uaccaaguuuauucugugagaua

SEQ ID NO: 272 - DNA, 23 bp, artificial
  mmu-miR-465a-5p
uauuuagaauggcacugauguga

SEQ ID NO: 273 - DNA, 22 bp, artificial
  mmu-miR-465b-5p
uauuuagaauggugcugaucug

SEQ ID NO: 274 - DNA, 22 bp, artificial
  mmu-miR-465c-5p
uauuuagaauggcgcugaucug

SEQ ID NO: 275 - DNA, 23 bp, artificial
  mmu-miR-468
uaugacugaugugcgugugucug

SEQ ID NO: 276 - DNA, 22 bp, artificial
  mmu-miR-98
ugagguaguaaguuguauuguu

SEQ ID NO: 277 - DNA, 22 bp, artificial
  mmu-miR-99a
aacccguagauccgaucuugug

SEQ ID NO: 278 - DNA, 22 bp, artificial
  mmu-miR-99b
cacccguagaaccgaccuugcg

SEQ ID NO: 279 - DNA, 21 bp, artificial
  old mmu-miR-422b
cuggacuuggagucagaaggc

SEQ ID NO: 280 - DNA, 21 bp, artificial
  miR-106b
uaaagugcugacagugcagau

SEQ ID NO: 281 - DNA, 23 bp, artificial
  miR-20b
caaagugcucauagugcagguag

SEQ ID NO: 282 - DNA, 23 bp, artificial
  miR-17
caaagugcuuacagugcagguag

SEQ ID NO: 283 - DNA, 22 bp, artificial
  miR-291a
caucaaaguggaggcccucucu

SEQ ID NO: 284 - DNA, 22 bp, artificial
  miR-291b-5p
gaucaaaguggaggcccucucc

SEQ ID NO: 285 - DNA, 22 bp, artificial
  miR-25
cauugcacuugucucggucuga

SEQ ID NO: 286 - DNA, 22 bp, artificial
  miR-32
uauugcacauuacuaaguugca

SEQ ID NO: 287 - DNA, 21 bp, artificial
  miR-92a-1
uauugcacuugucccggccug

SEQ ID NO: 288 - DNA, 22 bp, artificial
  miR-92a-2
uauugcacucgucccggccucc

SEQ ID NO: 289 - DNA, 22 bp, artificial
  miR-92b
uauugcacucgucccggccucc

SEQ ID NO: 290 - DNA, 22 bp, artificial
  miR-367
aauugcacuuuagcaaugguga

SEQ ID NO: 291 - DNA, 23 bp, artificial
  miR-19b
ugugcaaauccaugcaaaacuga

SEQ ID NO: 292 - DNA, 22 bp, artificial
  miR-290-5p
acucaaacuaugggggcacuuu

SEQ ID NO: 293 - DNA, 22 bp, artificial
  miR-292
acucaaacugggggcucuuuug

SEQ ID NO: 294 - DNA, 23 bp, artificial
  miR-200c
uaauacugccggguaaugaugga

SEQ ID NO: 295 - DNA, 23 bp, artificial
  miR-20a
uaaagugcuuauagugcagguag

SEQ ID NO: 296 - DNA, 22 bp, artificial
  miR-291b-3p
aaagugcauccauuuuguuugu

(19) United States
(12) Patent Application Publication
(10) Pub. No.: US 2015/0232810 A1
(43) Pub. Date: Aug. 20, 2015

(54) METHODS OF PREPARING PLURIPOTENT STEM CELLS

(71) Applicant: Stemgent, Inc., Cambridge, MA (US)

(72) Inventors: Chenmei Luo, Shrewsbury, MA (US); Kerry Mahon, Palo Alto, CA (US); Jonathon Bradley Hamilton, Littleton, MA (US)

(21) Appl. No.: 14/567,968

(22) Filed: Dec. 11, 2014

Related U.S. Application Data
(63) Continuation of application No. PCT/US13/45686, filed on Jun. 13, 2013.
(60) Provisional application No. 61/659,240, filed on Jun. 13, 2012.

(51) Int. Cl. C12N 5/074; A61K 35/545; A61K 35/51; A61K 35/36; A61K 35/44; G01N 33/50; A61K 35/33

(52) U.S. Cl. CPC C12N 5/0696; G01N 33/5008; G01N 33/5014; A61K 35/545; A61K 35/33; A61K 35/36; A61K 35/44; A61K 35/51; C12N 2500/40; C12N 2500/02; C12N 2501/65; C12N 2501/602; C12N 2501/603; C12N 2501/604; C12N 2501/606; C12N 2501/608; C12N 2501/605; C12N 2501/60

(57) ABSTRACT

The invention relates to pluripotent stems cells and their methods of use. The invention also relates to methods of producing pluripotent stem cells.

### CROSS REFERENCE

This application claims priority to U.S. Provisional Application No. 61/659,240 filed Jun. 13, 2013, herein incorporated by reference in its entirety.

### FIELD OF THE INVENTION

The invention relates to methods of preparing pluripotent stem cells and their method of use.

### BACKGROUND OF THE INVENTION

The widespread adoption of induced pluripotent stem (iPS) cell technology for regenerative medicine and drug screening applications has been limited by the inability to efficiently derive human iPS cell lines that are free from both genomic perturbation and viral contaminants.

iPS cells were first described by Yamanaka in 2006 (Cell 2006 126(4):663-676) and were immediately recognized for their potential to revolutionize the field of personalized medicine. Yamanaka describe the results of experiments, first performed in mice and then in human cells, wherein the addition of four transcription factors (reprogramming factors), Oct4, Sox2, Klf4 and c-Myc, to a fibroblast led to the de-differentiation of the somatic fibroblast cell to a cell in a pluripotent state.

Early methods of generating iPS cells focused on the use of retroviruses for delivering the reprogramming factors. Such viruses require significant safety precautions when handling, and their mode of action requires integration of the virus into the host cell genome to express the encoded transcription factor. DNA-based methods of generating iPS cells have also been developed and, although these methods are safer than retrovirus based methods, with regards to handling, these methods carry a risk of homologous recombination with the host cell genome. Both viral and DNA based methods of generating iPS cells therefore cannot be used to produce clinical grade cells. Further, iPS cells produced by viral and DNA based methods must be extensively screened prior to use to ensure that any genomic modifications that may have occurred do not affect the function of the cells.

Recent developments using messenger RNA (mRNA) to generate induced pluripotent stem (iPS) cells have led to improved methods of producing iPS cells. However, certain cell lines remain refractory to reprogramming with mRNA. Further, methods of generating iPS cells generate only a small number of iPS colonies per culture. mRNA based methods for producing iPS cells require multiple transfections (for example, a culture must be transfected every day for 16-18 days) and often requires growth on a feeder layer of cells.

There is a long felt need for a method of preparing pluripotent stem cells that can be rapidly prepared, for example from primary patient cells, do not require a step of screening for genetic modifications, and are safe to use for clinical applications. The novel methods of preparing pluripotent stems cells of the invention wherein a combination of mRNA and miRNA is introduced into the cells, as well as the cells themselves, satisfy this long felt need.

### BRIEF SUMMARY OF THE INVENTION

The present invention is directed to a novel method of generating iPS cells wherein cells are combined with a combination of mRNA and miRNA. The method of the invention offers numerous surprising and unexpected advantages as compared to methods of producing iPS cells known in the art, including virus based methods, DNA based methods and mRNA based methods that do not utilize miRNA.

The claimed method of producing iPS cells by the addition of microRNA (miRNA) in combination with mRNA improves upon any known methods of producing iPS cells because it provides for: 1) faster kinetics for the reprogramming process as compared to any known methods; and 2) higher productivity as compared to any known methods.

A decrease in the amount of time required to produce iPS cells is clearly an advantage. The novel claimed method of producing iPS cells of the invention also offers the advantage of requiring significantly fewer transfections, as compared to any known method of producing iPS cells.

The novel method of the invention also provides for production of an increased number of iPS cell colonies from typical patient lines as compared to other methods. Further, the claimed method of producing iPS cells of the invention enables the generation of iPS cells from cells that have not yielded any colonies when subjected to any known method of producing iPS cells.

Unlike virus based methods of producing iPS cells the novel claimed method is safe and provides an efficient method for producing iPS cells suitable for clinical use. Differentiated progeny cells derived from iPS cells of the invention are also suitable for clinical use. The claimed method can be performed without the need for any significant safety precautions. Unlike iPS cells prepared by methods that require viral vectors, iPS cells produced by the novel method of the invention offer the advantage of being free from viral contaminants and therefore are suitable for clinical applications. iPS cells and differentiated progeny cells produced from the iPS cells, such as those produced by the claimed methods, are advantageous over cells produced by other methods because they can be used for the development of personalized treatments and for regenerative medicine applications.

The claimed methods provide for a method of producing iPS cells wherein there is no risk of the occurrence of homologous recombination with the host cell genome. The claimed method produces iPS cells that have no genomic integrations and therefore require no pre-screening to determine genomic modifications as do iPS cells prepared by other methods known in the art.

Unlike art-accepted methods of producing iPS cells, the claimed method eliminates inherent variability associated with feeder based reprogramming methods by pairing a defined, xeno-free cell culture medium (that is, the medium contains no non-human components) with pluripotent cell culture attachment substrates. In addition, according to the claimed methods, a reduced number of transfections are ultimately required to establish iPS cell colonies. Further, a reduced amount of mRNA is needed per daily transfection.

Unlike iPS cells produced by methods currently known in the art, the claimed method offers the advantage of producing iPS cells that can be banked and used for experiments 4-5 weeks or more following production.

In addition, unlike other methods, the claimed method does not require post-colony isolation screening for genomic integrations or viral contaminants.

The methods of preparing pluripotent stem cells of the current invention clearly provide at least the following advantages over other methods known in the art: the use of mRNA and miRNA allows for fine control of stoichiometry and expression levels; the use of mRNA and miRNA allows for temporal control of stoichiometry and expression levels; because there is no integration of either mRNA or miRNA the method of the invention is suitable for the production of clinically relevant cells as compared to methods known in the art which use virus and therefore create a safety concern for clinical use; the timeline for colony formation, identification and isolation can be under 14 days according to the methods described herein, as compared to prior art methods that may require 40 days or more for production of pluripotent stem cells; the methods described herein do not require reseeding the cells although reseeding can be done, in contrast to methods known in the art that require reseeding after viral transduction; and the method is performed in the absence of a feeder layer as compared to methods known in the art that require the use of a feeder layer.

The effect on cell reprogramming is to enhance reprogramming. The methods of the present invention include inducing pluripotency in a cell, such that the cell becomes capable of dividing and differentiating into any cell type other than embryonic cells. Cellular reprogramming also induces de-differentiation of a cell. Altering cell reprogramming can enhance the level of pluripotency or de-differentiation that has been induced by an agent other than the combination of mRNA and microRNA. The pluripotent or multipotent cells, also called stem cells, have the ability to divide (self-replicate or self-renew) or differentiate into multiple different phenotypic lineages for indefinite periods. The cells of the present invention, under specific conditions, or in the presence of optimal regulatory signals, can become pluripotent and differentiate themselves into many different cell types that make up the organism.

The pluripotent or multipotent cells of the present invention possess the ability to differentiate into cells that have characteristic attributes and specialized functions, such as hair follicle cells, blood cells, heart cells, eye cells, skin cells, pancreatic cells, or nerve cells. In particular, pluripotent cells of the invention can differentiate into multiple cell types including but not limited to: cells derived from the endoderm, mesoderm or ectoderm, including but not limited to cardiac cells, neural cells (for example, astrocytes and oligodendrocytes), hepatic cells (for example, pancreatic islet cells), osteogentic, muscle cells, epithelial cells, chondrocytes, adipocytes, dendritic cells and, haematopoietic and retinal pigment epithelial (RPE) cells.

iPS cells are promising tools for the treatment of neurodegenerative disorders. For example, somatic cells from a patient with a disorder can be transformed into iPS cells using the methods of the invention and further differentiated to the desired neural subtype. Such cells can then be used in the development of disease models for the discovery of new compounds or other agents capable of treating the disease and/or for treating compounds used for therapy. In certain cases, the differentiated cells can be used for cell therapy to replace damaged tissue.

Examples of differentiation methods to the neural subtypes motor neuron and dopaminergic neuron and their application to the development of new therapies are found in the following references: Saporta et al. “Induced pluripotent stem cells in the study of neurological diseases” Stem Cell Research & Therapy 2011, 2:37; Lopez-Gonzalez, R. and Velasco, 1. “Therapeutic; Potential of Motor Neurons Differentiated from Embryonic Stem Cells and Induced Pluripotent Stem Cells” Arch Med Res 2012, 43:1, 1-10; Cooper et al. “Differentiation of human ES and Parkinson's disease iPS cells into ventral midbrain dopaminergic neurons requires a high activity form of SHH, FGF8a and specific regionalization by retinoic acid” Molecular and Cellular Neuroscience 45 (2010) 258-266; Dims et al., “Induced Pluripotent Stem Cells Generated from Patients with ALS Can Be Differentiated into Motor Neurons” Science 2008 321, 1218; Hu et al., “Neural differentiation of human induced pluripotent stem cells follows developmental principles but with variable potency” Proc. Nat. Acad. Sci. USA 2010, 107, 9, 4335; Mohamad O, Drury-Stewart D, Song M, Faulkner B, Chen D, et al. (2013) Vector-Free and Transgene-Free Human iPS Cells Differentiate into Functional Neurons and Enhance Functional Recovery after Ischemic Stroke in Mice. PLoS ONE 8(5): e64160. doi:10.1371/journal.pone.0064160; Osadaka et al., “Control of neural differentiation from pluripotent stem cells” Inflammation and Regeneration 2008 Vol. 28 No. 3 166; Marchetto et al., “Induced pluripotent stem cells (iPSCs) and neurological disease modeling: progress and promises” Human Molecular Genetics, 2011, Vol. 20, Review Issue 2 R109-R115.

Methods of differentiating stem cells are well known in the art and include, for example, contacting pluripotent stem cells with appropriate growth factors and/or cytokines.

Cell reprogramming can further include partial de-differentiation to a closely related cell or cell type and/or trans-differentiation, wherein a cell of the present invention converts from one differentiated cell type into another differentiated cell type.

Moreover, to enhance the efficiency to establish induced pluripotent stem (iPS) cells, the following cytokines and/or small molecules, in addition to the abovementioned miRNAs, may further be introduced into somatic cells to be reprogrammed: i.e., basic fibroblast growth factor (bFGF), stem cell factor (SCF), etc. for the cytokines; and histone deacetylase inhibitors such as valpronic acid, DNA methylase inhibitors such as 5′-azacytidine, histone methyltransferase (G9a) inhibitors such as BIX01294 (BIX), etc. for the small molecules (D. Huangfu et al., Nat. Biotechnol., 26, pp. 795-797, 2008; S. Kubicek et al., Mol. Cell, 25, pp. 473-481, 2007; Y. Shi et al., Cell Stem Cell, 3, 568-574, 2008, Yan Shi et al., Cell Stem Cell, 2, pp. 525-528, 2008. In addition, p53 inhibitors such as shRNA or siRNA for p53 and/or UTF1 may be introduced into somatic cells (Yang Zhao et al., Cell Stem Cell, 3, pp 475-479, 2008). Also, activation of the Wnt signal (Marson A. et al., Cell Stem Cell, 3, pp 132-135, 2008) or inhibition of signaling by mitogen-activated protein kinase or glycogen synthase kinase-3 (Silva J. et al., PoS Biology, 6, pp 2237-2247 2008) can serve as a means for increasing the efficiency of generating iPS cells.

The invention provides for a method of producing a pluripotent stem cell comprising: introducing at least one mRNA into a target cell; introducing at least one miRNA into a target cell; and culturing the target cell to produce a pluripotent stem cell.

The step of introducing the at least one mRNA into the cell and or the step of introducing the at least one miRNA into the target cell can be repeated at least once.

Prior to step (a), at least one miRNA can be introduced into the target cell.

Steps (a) and (b) can be sequential.

Steps (a) and (b) can occur simultaneously.

The stem cell can be produced in less than 2 weeks from the initiation of step (a), for example, 13 days, 12 days, 11 days, 10 days, 9 days, 8 days, 7 days, 6 days, 5 days or less from the initiation of step (a). Alternatively, a stem cell of the invention is produced in more than 2 weeks, for example 2-10 weeks, 2-5 weeks and 2-3 weeks from the initiation of step (a).

The stem cell that is produced can express at least one of a surface marker selected from the group consisting of: SSEA3, SSEA4, Tra-1-81, Tra-1-60, Rex1, Oct4, Nanog and Sox2.

The stem cells can divide in vitro for greater than one year; and/or divide in vitro for more than 30 passages; and/or stain positive by alkaline phosphatase or Hoechst Stain, and/or form a teratoma.

The stem cell can form an embryoid body and express one or more endoderm markers selected from the group consisting of: AFP, FOXA2 and GATA4, and/or one or more mesoderm markers selected from the group consisting of: CD34, CDH2 (N-cadherin), COL2A1, GATA2, HAND1, PECAM1, RUNX1, RUNX2; and/or one or more ectoderm markers selected from the group consisting of: ALDH1A1, COL1A1, NCAM1, PAX6 and TUBB3 (Tuj1).

At least 1 stem cell is produced, for example, 1, 2, 3, 4, 5, 6, 7, 8, 9, 10, 15, 20, 25, 30, 35, 40, 45, 50, 60, 70, 80, 90, 100, 500, 1000 or more.

One or both of the at least one miRNA and the at least one mRNA can comprise at least one modified nucleotide as defined herein.

Neither of the at least one miRNA and the at least one mRNA are provided in a DNA vector or a viral vector.

One or both of the at least one miRNA and the at least one mRNA can comprise a modified nucleotide, for example 5-methylcytosine or pseudouracil, or any modified nucleotide as defined herein.

The at least one mRNA is not integrated into the genome of the stem cell.

The mRNA and miRNA introduced into the target cells in steps (a) and (b) are not present in the stem cell.

The culturing can be performed in the absence of a feeder layer.

The method can be performed at ≦5% O₂.

The method can be performed at 5%-21% O₂, for example, 6, 7, 8, 9, 10, 15, 20 and 21%, for example, at 21% O₂

The target cell includes but is not limited to fibroblasts, peripheral blood derived cell types (specifically late—endothelial progenitor cell (L-EPCs)), cord blood derived cell types (CD34+), epithelial cells and keratinocyte.

The at least one mRNA encodes a reprogramming factor.

The at least one mRNA can encode at least one of OCT4, SOX2, KLF4, c-MYC and LIN28.

The at least one miRNA can comprise at least one miRNA that is 80% or more identical to an miRNA selected from the group consisting of hsa-miR-302a, hsa-miR-302b, hsa-miR-302c, hsa-miR302d, hsa-miR367, hsa-miR-200c, hsa-miR-369-3p and hsa-miR-369-5p.

The at least one miRNA can also comprise a combination of hsa-miR-302a, hsa-miR-302b, hsa-miR-302c, hsa-miR302d and hsa-miR367.

The at least one miRNA can also comprise a combination of hsa-miR-302a, hsa-miR-302b, hsa-miR-302c, hsa-miR302d, hsa-miR-200C, hsa-miR-369-3p and hsa-miR-369-5p.

The at least one miRNA can also comprise a combination of hsa-miR-302a, hsa-miR-302b, hsa-miR-302c, hsa-miR302d, hsa-miR-200c, hsa-miR-369-3p and hsa-miR-369-5p.

The at least one miRNA can comprise the combination of: hsa-miR-302a, hsa-miR-302b, hsa-miR-302c, hsa-miR-302d and hsa-miR-367; or hsa-miR-302a, hsa-miR-302b, hsa-miR-302c, hsa-miR-302d, hsa-miR-200C, hsa-miR-369-3p, hsa-miR-369-5p; or the combination of hsa-miR-302a, hsa-miR-302b, hsa-miR-hsa-miR-302c, hsa-miR-302d, hsa-miR-200C, hsa-miR-369-3p, hsa-miR-369-5p

The target cell can be a mammalian cell, including but not limited to a human cell.

The invention also provides for a method of inducing pluripotency in a target cell comprising: introducing at least one mRNA into the target cell; introducing at least one miRNA into the target cell; and culturing the target cell to produce a pluripotent cell.

The invention also provides for an isolated pluripotent stem cell comprising at least one mRNA encoding a reprogramming factor in combination with at least one miRNA produced according to any one of the methods described herein.

The invention also provides for a formulation comprising the isolated pluripotent stem cell as defined herein and produced by any one of the methods described herein, or a differentiated cell derived from an isolated pluripotent stem cell as defined herein, for example, in combination with a pharmaceutical carrier.

The formulation can further comprise a compound that suppresses an immune response.

As used herein, compound includes any one of a protein, an antibody, a nucleic acid, for example, siRNA, miRNA, antisense RNA, mRNA and/or a small molecule.

The invention also provides for a kit for producing a pluripotent stem cell or a differentiated progeny cell comprising at least one mRNA and at least one miRNA.

The kit can further comprise culture media and/or a transfection reagent.

The kit can further comprise a compound that suppresses an immune response.

The invention also provides for a method of treating a subject with any of the diseases described herein comprising administering to the subject the isolated pluripotent stem cell of the invention and produced by any of the methods described herein.

The invention also provides for a method of treating a subject with any of the diseases described herein, comprising administering to the subject a progeny cell produced by differentiation of the isolated pluripotent stem cell obtained by the methods of the invention.

The invention also provides for a method of identifying a compound for treatment of a disease comprising contacting a cell produced by differentiation of a stem cell produced by the methods of the invention with a compound of interest.

The invention also provides for a method of determining the activity of a compound for treating a disease comprising contacting a cell produced by differentiation of a stem cell produced by the methods of the invention with a compound known to treat a disease.

The invention also provides a method of determining the toxicity of a compound for treating a disease comprising contacting a cell produced by differentiation of a stem cell produced by the methods of the invention with a compound known to treat a disease.

According to the methods of the invention, the cell produced by differentiation of a stem cell is selected from the group consisting of: fibroblast, peripheral blood derived cells including but not limited to endothelial progenitor cell (L-EPCs)), cord blood derived cell types (CD34+), epithelial cells, and keratinocytes.

The invention also provides for the use of a cell produced by differentiation of a stem cell produced by the methods of the invention for the manufacture of a medicament for treating a subject with a disease.

### BRIEF DESCRIPTION OF THE DRAWINGS

FIGS. 1A-C present the results of transfections of fibroblasts with eGFP mRNA (A: fluorescence intensity as determined by flow cytometry; B: representative histograms; C: fluorescent imaging of cells transfected with eGFP mRNA).

FIGS. 2A-B present A: the timeline for production of iPS cells from primary patient fibroblasts; and B: the morphology progression during iPS cell production.

FIG. 3 presents the effect of target cell number and mRNA dose on iPS cell generation.

FIG. 4 presents a graph demonstrating the number of mRNA transfections required to generate Tra-1-81(+) iPS cell colonies.

FIG. 5 presents results demonstrating that iPS cell colonies can be formed when cells are treated with miRNA in the presence of low levels of mRNA.

FIG. 6 demonstrates the continued expansion and maintenance of pluripotency of clonal mRNA iPS cells lines under feeder free conditions.

FIG. 7 presents morphological progression of an iPS colony from cells refractory to other methods of reprogramming.

FIGS. 8A-B present the nucleotide sequence (A) and amino acid sequence (B) of OCT4.

FIGS. 9A-B present the nucleotide sequence (A) and amino acid sequence (B) of SOX2.

FIG. 10 presents the amino acid sequence of NANOG.

FIGS. 11A-B present the nucleotide sequence (A) and amino acid sequence (B) of LIN28.

FIGS. 12A-B present the nucleotide sequence (A) and amino acid (B) sequence of KLF4 FIGS. 13A-C present the nucleotide sequence (A-B) and amino acid sequence (C) of cMYC.

FIG. 14 presents the nucleotide sequence of GFP.

### DETAILED DESCRIPTION OF THE INVENTION

### Definitions

As used herein, “pluripotent” as it refers to a “pluripotent stem cell” means a cell with the developmental potential, under different conditions, to differentiate to cell types characteristic of all three germ cell layers, i.e., endoderm (e.g., gut tissue), mesoderm (including blood, muscle, and vessels), and ectoderm (such as skin and nerve). Pluripotent cell as used herein, includes a cell that can form a teratoma which includes tissues or cells of all three embryonic germ layers, or that resemble normal derivatives of all three embryonic germ layers (i.e., ectoderm, mesoderm, and endoderm) are formed. A pluripotent cell of the invention also means a cell that can form an embryoid body (EB) and express markers for all three germ layers including but not limited to the following: endoderm markers-AFP, FOXA2, GATA4; mesoderm markers-CD34, CDH2 (N-cadherin), COL2A1, GATA2, HAND1, PECAM1, RUNX1, RUNX2; and Ectoderm markers-ALDH1A1, COL1A1, NCAM1, PAX6, TUBB3 (Tuj1).

A pluripotent cell of the invention also means a human cell that expresses at least one of the following markers: SSEA3, SSEA4, Tra-1-81, Tra-1-60, Rex1, Oct4, Nanog, Sox2 as detected using methods known in the art. A pluripotent stem cell of the invention includes a cell that stains positive with alkaline phosphatase or Hoechst Stain.

A pluripotent cell has a lower developmental potential than a totipotent cell. The ability of a cell to differentiate to all three germ layers can be determined using, for example, a nude mouse teratoma formation assay. In some embodiments, pluripotency can also be evidenced by the expression of embryonic stem (ES) cell markers. Pluripotency of a cell or population of cells generated using the compositions and methods described herein is also determined by the developmental potential to differentiate into cells of each of the three germ layers.

In some embodiments, a pluripotent cell is termed an “undifferentiated cell.” Accordingly, the terms “pluripotency” or a “pluripotent state” as used herein refer to the developmental potential of a cell that provides the ability of the cell to differentiate into all three embryonic germ layers (endoderm, mesoderm and ectoderm). Those of skill in the art are aware of the embryonic germ layer or lineage that gives rise to a given cell type. A cell in a pluripotent state typically has the potential to divide in vitro for a long period of time, e.g., greater than one year or more than 30 passages.

As used herein, the term “induced pluripotent stem cells (iPS cells)” refers to cells having similar properties to those of ES cells. In particular, an “iPS” cell as used herein, includes an undifferentiated cell which is reprogrammed from somatic cells and have pluripotency and proliferation potency. However, this term is not to be construed as limiting in any sense, and should be construed to have its broadest meaning. As used herein, the term “pluripotent stem cell”, as it refers to the cell produced by the claimed methods is synonymous with the term “iPS”. iPS cells of the invention are generated from a variety of cell types including but not limited to fibroblasts, peripheral blood derived cell types (specifically late—endothelial progenitor cell (L-EPCs)), cord blood derived cell types (CD34+), epithelial cells and keratinocytes.

The invention also provides for colonies of iPS cells produced, for example, by providing a non-pluripotent cell (somatic), culturing this cell in a media, culturing this cell on a surface, culturing this cell with a feeder cell (for example, NuFF or MEF-mouse embryonic fibroblast) introducing mRNA, introducing miRNA, introducing mRNA and miRNA, optionally splitting the cell culture, identifying stem cell colonies using surface markers or morphology, isolating the colony, and subculturing the isolated colony. A pluripotent stem cell colony will exhibit some or all of the characteristics described above for pluripotent stem cells.

As used herein, the term “somatic cell” also refers to any cell other than a germ cell, a cell present in or obtained from a pre-implantation embryo, or a cell resulting from proliferation of such a cell in vitro. Stated another way, a somatic cell refers to any cell forming the body of an organism, as opposed to a germline cell. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated, pluripotent, embryonic stem cells—is a somatic cell: internal organs, skin, bones, blood, and connective tissue are all made up of somatic cells.

In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro. Unless otherwise indicated, the compositions and methods for reprogramming a somatic cell described herein can be performed both in vivo and in vitro (where in vivo is practiced when a somatic cell is present within a subject, and where in vitro is practiced using an isolated somatic cell maintained in culture).

As used herein, the term “reprogramming factor,” refers to factor that can alter the developmental potential of a cell, such as a protein, an RNA, or a small molecule, the expression of which contributes to the reprogramming of a cell, e.g. a somatic cell, to a less differentiated or undifferentiated state, e.g. to a cell of a pluripotent state or partially pluripotent state. A reprogramming factor can be, for example, transcription factors that can reprogram cells to a pluripotent state, such as, but not limited to, SOX2, OCT3/4, KLF4, NANOG, LIN-28, c-MYC, Glis1, Sal4, Esrbb1 and the like, including but not limited to, any gene, protein, RNA or small molecule, that can substitute for one or more of these transcription factors in a method of reprogramming cells in vitro.

The term “cell reprogramming” refers to altering the natural state of the cell such that the cell becomes pluripotent and is capable of dividing and differentiating into any cell type other than embryonic cells. Cellular reprogramming can include inducing pluripotency in or de-differentiation of the cell. Altering cell reprogramming can also refer to enhancing the level of pluripotency or de-differentiation that has been induced by an agent other than a microRNA. Pluripotent or multipotent cells, also called stem cells, have the ability to divide (self-replicate or self-renew) or differentiate into multiple different phenotypic lineages for indefinite periods; in some cases throughout the life of the organism. A stem cell population is a population that possesses at least one stem cell. When pluripotent stem cells are derived from a non-pluripotent cell, such as for example a somatic cell, they are termed induced pluripotent stem cells (iPS or iPSCs). Cell reprogramming can further include partial de-differentiation to a closely related cell or cell type. Cell reprogramming can also include trans-differentiation. Trans-differentiation is defined as the conversion of one differentiated cell type into another, such as for example conversion of exocrine cells into beta-islet-like cells. (See, e.g., Blelloch, et al., Short cut to cell replacement, Nature, 455:604-605 (2008).) The term “progenitor cell” is used herein to refer to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, “nucleotide” is used as recognized in the art to include those with natural bases (standard), and modified bases well known in the art. The nucleotides can be unmodified or modified at the sugar, phosphate and/or base moiety, (also referred to interchangeably as nucleotide analogs, modified nucleotides, non-natural nucleotides, non-standard nucleotides and other; see, e.g., Usman and McSwiggen, supra; Eckstein, et al., International PCT Publication No. WO 92/07065; Usman et al, International PCT Publication No. WO 93/15187; Uhlman & Peyman, supra, all are hereby incorporated by reference herein). Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine and pseudouridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

As used herein, the term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide.

By “RNA” is meant a molecule comprising at least one ribonucleotide residue. By “ribonucleotide” is meant a nucleotide with a hydroxyl group at the 2′ position of a 3-D-ribofuranose moiety. The term RNA includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

As used herein, “modified nucleotide” refers to a nucleotide that has one or more modifications to the nudeoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-O-methyl, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA, and 2′-O-(N-methylcarbamate) or those comprising base analogs. In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878.

As used herein, “microRNA” or “miRNA” refers to a nucleic acid that forms a single-stranded RNA, which single-stranded RNA has the ability to alter the expression (reduce or inhibit expression; modulate expression; directly or indirectly enhance expression) of a gene or target gene when the miRNA is expressed in the same cell as the gene or target gene. In one embodiment, a miRNA refers to a nucleic acid that has substantial or complete identity to a target gene and forms a single-stranded miRNA. In some embodiments miRNA may be in the form of pre-miRNA, wherein the pre-miRNA is double-stranded RNA. The sequence of the miRNA can correspond to the full length target gene, or a subsequence thereof. Typically, the miRNA is at least about 15-50 nucleotides in length (e.g., each sequence of the single-stranded miRNA is 15-50 nucleotides in length, and the double stranded pre-miRNA is about 15-50 base pairs in length). In some embodiments the miRNA is 20-30 base nucleotides. In some embodiments the miRNA is 20-25 nucleotides in length. In some embodiments the miRNA is 20, 21, 22, 23, 24, 25, 26, 27, 28, 29, or 30 nucleotides in length.

The invention also provides for pluripotent stem cells that are produced by introducing into the cells a combination of mRNA and miRNA mimics. As used herein, the term, “miRNA mimic” means synthetic miRNA that has enhanced stability due to modified nucleotides or structural modifications (e.g. bulges or loops). As used herein, the term “miRNA mimic” also means small, chemically modified double-stranded RNAs that mimic endogenous miRNAs and enable miRNA functional analysis by up-regulation of miRNA activity. They are typically hairpins, for example, formed by single stranded miRNA that forms a double stranded portion that is a hairpin loop.

The term “contacting” or “contact” as used herein in connection with contacting a cell with one or more mRNAs or miRNAs as described herein, includes subjecting a cell to a culture medium which comprises one or more mRNAs or miRNAs at least one time, or a plurality of times, or to a method whereby such mRNAs and/or miRNAs are forced to contact a cell at least one time, or a plurality of times, i.e., a transfection system. Preferably, the mRNA and miRNA, when introduced into a cell, are not present in a DNA or viral vector. mRNA and miRNA of the invention that are not in a DNA or viral vector can be introduced or transfected into a cell according to methods known in the art, for example, electroporation and lipofection.

As used herein, the term “transfection reagent” refers to any agent that induces uptake of a synthetic, mRNA or miRNA into a host cell. Also encompassed are agents that enhance uptake e.g., by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 25-fold, at least 500-fold, at least 100-fold, at least 1000-fold, or more, compared to an mRNA or miRNA administered in the absence of such a reagent. In one embodiment, a cationic or non-cationic lipid molecule useful for preparing a composition or for co-administration with an mRNA or miRNA is used as a transfection reagent. In other embodiments, the mRNA or miRNA comprises a chemical linkage to attach e.g., a ligand, a peptide group, a lipophilic group, a targeting moiety etc. In other embodiments, the transfection reagent comprises a charged lipid, an emulsion, a liposome, a cationic or non-cationic lipid, an anionic lipid, or a penetration enhancer as known in the art or described herein.

As used herein, the term “repeated transfections” refers to repeated transfection of the same cell culture with an mRNA or miRNA of the invention, a plurality of times (e.g., more than once or at least twice). In some embodiments, the cell culture is transfected at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times at least 18 times, at least 19 times, at least 20 times, at least 25 times, at least 30 times, at least 35 times, at least 40 times, at least 45 times, at least 50 times or more. The transfections can be repeated until a desired phenotype of the cell is achieved.

The time between each repeated transfection is referred to herein as the “frequency of transfection.” In some embodiments, the frequency of transfection occurs every 6 h, every 12 h, every 24 h, every 36 h, every 48 h, every 60 h, every 72 h, every 96 h, every 108 h, every 5 days, every 7 days, every 10 days, every 14 days, every 3 weeks, or more during a given time period in any method of producing a pluripotent stem cell or any method of inducing pluripotency in a cell according to the invention. The frequency can also vary, such that the interval between each dose is different (e.g., first interval 36 h, second interval 48 h, third interval 72 h etc). It should be understood depending upon the schedule and duration of repeated transfections, it will often be necessary to split or passage cells or change or replace the media during the transfection regimen to prevent overgrowth and replace nutrients. For the purposes of the methods described herein, transfections of a culture resulting from passaging an earlier transfected culture is considered “repeated transfection,” “repeated contacting” or “contacting a plurality of times,” unless specifically indicated otherwise.

The term “introducing” when used in the context of “introducing” an miRNA or mRNA into a cell refers to any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one miRNA into the host cell.

A variety of different types of cells can be utilized for the methods of the present invention. Cells that may express an mRNA and/or miRNAs of the invention can include, e.g., fibroblast cells, peripheral blood derived cells including but not limited to endothelial progenitor cell (L-EPCs)), cord blood derived cell types (CD34+), epithelial cells, and keratinocytes.

The cells can be any of the cells typically utilized in generating cells that harbor recombinant nucleic acid constructs. Cells useful according to the methods of the invention include, but are not limited mouse embryonic fibroblasts (MEFs). The cells can be mammalian cells, for example, human, rodent or primate. Cell types utilized for the methods of the present invention can also include cells from tissue samples including but not limited to blood, bone, brain, kidney, muscle, spinal cord, nerve, endocrine system, uterine, ear, foreskin, liver, intestine, bladder or skin, for example, as derived from a subject diagnosed with a particular disease or in need of pluripotent stem cells. The cells can include neural cells, lymphocytes, epidermal cells, islet cells, intestinal cells or fibroblasts. The cells of the present invention can be autologous or heterologous cells. The cells useful for the methods of the present invention can include animal cells. In some embodiments the cells are mammalian. In some embodiments the cell are from rodents or primates. In some embodiments the cells are mouse cells. In some embodiments are pig cells.

The types of target or somatic cells to be used for the formation of pluripotent stem cells of the invention or reprogrammed by the method of the present invention are not specifically limited, and any somatic cells can be used. For example, various somatic cells such as (1) tissue stem cells, e.g., neural stem cells, hematopoietic stem cells, mesenchymal stem cells, and dentis stem cells; (2) tissue precursor cells; and (3) differentiated cells, e.g., lymphocytes, epidermal cells, endothelial cells, muscle cells, fibroblast cells, pilary cells, skin cells, liver cells, gastric mucosa cells, intestine cells, spleen cells, pancreatic cells (including pancreatic exocrine cells), brain cells, lung cells, and renal cells can be reprogrammed. Blood cells including platelets, erytrocytes, leukocytes (neutrophils, eosinophils, basophils, lymphocytes, monocytes) and thrombocytes can be used to produce pluripotent stem cells according to the methods of the invention. For use of induced pluripotent stem cells or progeny cells differentiated from iPS cells in therapies against diseases, it is desirable to use somatic cells isolated from the patient. For example, somatic cells involved in a disease and somatic cells associated with a therapy for a disease can also be used.

As used herein “culture” means maintain for an appropriate amount of time under controlled conditions in a controlled and defined medium.

As used herein, “culture medium” means a medium optimized for mRNA based cellular reprogramming of human cells or a medium suitable for expanding and maintaining iPS cell lines. In one embodiment, a “culture medium” according to the invention is xeno-free. Culture medium useful according to the invention includes any medium known in the art to provide for production of pluripotent stem cells. Culture medium useful according to the invention also includes any medium known in the art to support maintenance of pluripotent stem cells. Culture medium according to the invention includes but is not limited to Pluriton™ Reprogramming Medium (Stemgent) for production of iPS cells, and Nutristem™ XF/FF Culture Medium (Stemgent) for maintenance of iPS cells.

By “subject” is meant an organism, which is a donor or recipient of explanted somatic cells or the pluripotent cells themselves. “Subject” also refers to an organism to which the pluripotent cells or differentiated progeny of the pluripotent cells of the invention can be administered. A subject can be a mammal or mammalian cells, including a human or human cells.

“Subject,” as used herein, is preferably, but not necessarily limited to, a human subject. The subject is male or female, and may be of any race or ethnicity. Subject as used herein may also include an animal, particularly a mammal such as a canine, feline, bovine, caprine, equine, ovine, porcine, rodent (e.g., a rat and mouse), a lagomorph, a primate (including non-human primate), etc., that may be treated in accordance with the methods of the present invention or screened for veterinary medicine or pharmaceutical drug development purposes. A subject according to some embodiments of the present invention includes a patient, human or otherwise, in need of therapeutic treatment for a disease according to the invention.

As used herein, “control subject” means a subject that has not been diagnosed with a disease according to the invention. A “control subject” also means a subject that is not at risk of developing a disease, as defined herein.

The phrase “pharmaceutically acceptable carrier” refers to a carrier for the administration of a therapeutic agent. Exemplary carriers include saline, buffered saline, dextrose, water, glycerol, ethanol, and combinations thereof.

Various methodologies of the instant invention include steps that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a method of producing a pluripotent stem cell or a method of inducing pluripotency, as described herein. For example, a transcription rate, mRNA level, translation rate, protein level, biological activity, cellular characteristic or property, genotype, phenotype, etc. can be determined prior to introducing an mRNA and miRNA of the invention into a cell or organism. In another embodiment, a “suitable control” or “appropriate control” is a value, level, feature, characteristic, property, etc. determined in a cell or organism, e.g., a control or normal cell or somatic cell or organism, exhibiting, for example, normal traits. In yet another embodiment, a “suitable control” or “appropriate control” is a predefined value, level, feature, characteristic, property, etc.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

“Treatment”, or “treating” as used herein, is defined as the application or administration of a pluripotent stem cell or a differentiated cell derived therefrom of the invention to a patient who has a disorder with the purpose to cure, heal, alleviate, relieve, alter, remedy, ameliorate, improve or affect the disease or disorder, or symptoms of the disease or disorder. The term “treatment” or “treating” is also used herein in the context of administering agents prophylactically. The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. The term “patient” includes human and other mammalian subjects that receive either prophylactic or therapeutic treatment.

As used herein, the term “biological sample” includes tissue; cultured cells, e.g., primary cultures, explants, and transformed cells; cellular extracts, e.g., from cultured cells, tissue, embryos, cytoplasmic extracts, nuclear extracts; blood, etc.

The term “autologous” when used herein designates host derived and transplanted re-inserted, re-administered or returned to the host from which the nucleic acid, protein, cell or tissue was derived.

A given miRNA sequence includes both the human and murine homologues or orthologs having structural and functional similarity to the referenced miRNA. The term, homolog applies to the relationship between genes separated by the event of speculation (ortholog) or to the relationship between genes separated by the event of genetic duplication (paralog). Orthologous miRNAs are miRNAs in different species that are similar to each other because they originated from a common ancestor. Homologous sequences are similar sequences which share a common ancestral DNA sequence or which would have been expected to share such given their high degree of sequence identity. Accordingly, in some embodiments, the ortholog or homologue is any sequence which differs from the sequence of the referenced miRNA by at most one, two or three nucleic acid residues.

An inhibitor of a miRNA can be an antisense nucleic acid or siRNA which is complementary to or shares substantial identity with the miRNA and can block the function of the miRNA.

As used herein, the term “substantial identity” refers to a sequence that hybridizes to a reference sequence under stringent conditions, or to a sequence that has a specified percent identity over a specified region of a reference sequence.

As used herein, the phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures.

An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Nucleic acids that do not hybridize to each other under stringent conditions are still substantially identical if the polypeptides which they encode are substantially identical. Those of ordinary skill will readily recognize that alternative hybridization and wash conditions can be utilized to provide conditions of similar stringency. Additional guidelines for determining hybridization parameters are provided in numerous references, e.g., and Current Protocols in Molecular Biology, ed. Ausubel, et al.

The terms “substantially identical” or “substantial identity,” in the context of two or more nucleic acids or polypeptide sequences, refer to two or more sequences or subsequences that are the same or have a specified percentage of amino acid residues or nucleotides that are the same (i.e., at least about 60%, preferably 65%, 70%, 75%, preferably 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 6-7 amino acids or 25 nucleotides in length, or more preferably over a region that is 50-100 amino acids or nucleotides in length, or the entire length.

For sequence comparison, typically one sequence acts as a reference sequence, to which test sequences are compared.

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention.

The term “administering,” as used herein, refers to any mode of transferring, delivering, introducing, or transporting an iPS cell or a differentiated progeny of the iPS of the invention to a subject. Such modes include, but are not limited to, oral, topical, intravenous, intraperitoneal, intramuscular, intradermal, intranasal, and subcutaneous administration. Preferably, administration is (1) intravenous, for example, wherein the iPS cells are contained in an IV bag or (2) via a medical device, for example, a stent, valve, balloon or a catheter, wherein the medical device is in combination with, or coated with, an iPS cell or iPS cell population of the invention.

In one embodiment, administration can be via an implantable or non-implantable drug delivery device in combination with an iPS cell or iPS cell population of the invention or via an implantable or non-implantable time release delivery device which may comprise a delivery device associated with the iPS cells of the invention.

By “ameliorate” is meant decrease, suppress, attenuate, diminish, arrest, or stabilize the development or progression of a disease.

By “delivering” is meant delivery of a therapeutic iPS cell or differentiated cell derived therefrom of the invention to a subject in need of treatment. For example, a therapeutic cell that has been differentiated from an iPS of the invention may be delivered to a vein, artery, capillary, heart, or tissue of a subject, as well as to a specific population, or sub-population, of cells. Delivery of a therapeutic cell of the invention may be assessed by adding tracking agents, such as gold, gadolinium, and/or the like, to the exosomes to allow identification of the tissues that take up the cells with MRI.

By “effective amount” or “therapeutically effective amount” is meant the amount of iPS cells or a population of iPS cells or differentiated cells derived from an iPS cell required to ameliorate the symptoms of a disease. By “effective amount” or “therapeutically effective amount” is also meant the amount of iPS cells or a population of iPS cells or differentiated cells derived therefrom, required to induce a therapeutic or prophylactic effect for use in therapy to treat a disease according to the invention. The effective amount of active compound(s), for example, cells of the invention, used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

“Disease,” “disorder,” and “condition” are commonly recognized in the art and designate the presence of signs and/or symptoms in an individual or patient that are generally recognized as abnormal and/or undesirable. Diseases or conditions may be diagnosed and categorized based on pathological changes.

As used herein, the terms “treat,” “treated,” “treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

A subject is said to be treated for a disease, if following administration of the cells of the invention, one or more symptoms of the disease are decreased or eliminated.

The cells of the invention, including differentiated progeny derived from iPS cells of the invention, are useful for treatment of a disease. In particular, any disease wherein cell therapy is appropriate can be treated using the iPS or differentiated progeny derived therefrom of the invention. Diseases where cell therapy is known in the art to be an appropriate method of therapy include but are not limited to: automimmune disease, diseases wherein treatment involves regeneration of neural cells/tissue, diseases wherein treatment involves regeneration of cardiac cells/tissues, Parkinson's Disease and Alzheimer's Disease. Cells differentiated from the iPS cells of the invention including myocardial cells, insulin producing cells or nerve cells can be safely utilized in stem cell transplantation therapies for treatment of various disease such as heart failure, insulin dependent diabetes mellitus, Parkinson's disease and spinal cord injury. iPS cells or differentiated cells derived therefrom can be used for autologous cells therapy, wherein the therapy is specific/personalized for a particular subject, for example to prevent an immune response, or non-autologous.

As used herein, the term “disease” includes any one or more of the following autoimmune diseases or disorders: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, and interstitial lung fibrosis.

In another embodiment, disease refers to any one of Wilson's disease, spinocerebellar ataxia, prion disease, Parkinson's disease, Huntington's disease, amytrophic lateral sclerosis, amyloidosis, Alzheimer's disease, Alexander's disease, alcoholic liver disease, cystic fibrosis, Pick's Disease, spinal muscular dystrophy or Lewy body dementia.

“Disease” also includes any one of rheumatoid spondylitis; post ischemic perfusion injury; inflammatory bowel disease; chronic inflammatory pulmonary disease, eczema, asthma, ischemia/reperfusion injury, acute respiratory distress syndrome, infectious arthritis, progressive chronic arthritis, deforming arthritis, traumatic arthritis, gouty arthritis, Reiter's syndrome, acute synovitis and spondylitis, glomerulonephritis, hemolytic anemia, aplastic anemia, neutropenia, host versus graft disease, allograft rejection, chronic thyroiditis, Graves' disease, primary binary cirrhosis, contact dermatitis, skin sunbums, chronic renal insufficiency, Guillain-Barre syndrome, uveitis, otitis media, periodontal disease, pulmonary interstitial fibrosis, bronchitis, rhinitis, sinusitis, pneumoconiosis, pulmonary insufficiency syndrome, pulmonary emphysema, pulmonary fibrosis, silicosis, or chronic inflammatory pulmonary disease.

“Disease” also refers to any one of cancer, tumor growth, cancer of the colon, breast, bone, brain and others (e.g., osteosarcoma, neuroblastoma, colon adenocarcinoma), chronic myelogenous leukemia (CML), acute myeloid leukemia (AML), acute promyelocytic leukemia (APL), cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

As used herein, “diagnosing” or “identifying a patient or subject having” refers to a process of determining if an individual is afflicted with a disease or ailment, for example a disease as defined herein.

Any compositions or methods provided herein can be combined with one or more of any of the other compositions and methods provided herein.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Reprogramming Factors

The term “factor” according to the invention when used in conjunction with the expression “reprogramming factor” thereof by RNA includes proteins and peptides as well as derivatives and variants thereof. For example, the term “reprogramming factor” includes but is not limited to: OCT4, SOX2, NANOG, LIN28, KLF4, c-MYC, L-Myc, Glis-1, Sal4, Esrbb1, LRH-1, RAR-gamma and any factor known in the art to have the ability to reprogram a cell as defined herein. The invention contemplates the use of any of the reprogramming factors described herein, either alone or in any combination.

The invention also contemplates the use of any of the following reprogramming factors: members of the Oct family, Kif family, Sox family, Myc family, Lin family, and Nanog family including, but are not limited to: Oct3/4 (also referred to as Oct3, Oct4 or POU5F1) for Oct family; Sox1, Sox2, Sox3, Sox4, Sox11 and Sox15 for Sox family; c-Myc, N-Myc and L-Myc for Myc family; Lin28 and Lin28b for Lin family; and Nanog for Nanog family.

The factors can be of any animal species; e.g., mammals and rodents.

OCT4 is a transcription factor of the eukaryotic POU transcription factors and an indicator of pluripotency of embryonic stem cells. It is a maternally expressed Octomer binding protein. It has been observed to be present in oocytes, the inner cell mass of blastocytes and also in the primordial germ cell. The gene POU5F1 encodes the OCT4 protein. Synonyms to the gene name include OCT3, OCT4, OTF3 and MGC22487. The presence of OCT4 at specific concentrations is necessary for embryonic stem cells to remain undifferentiated.

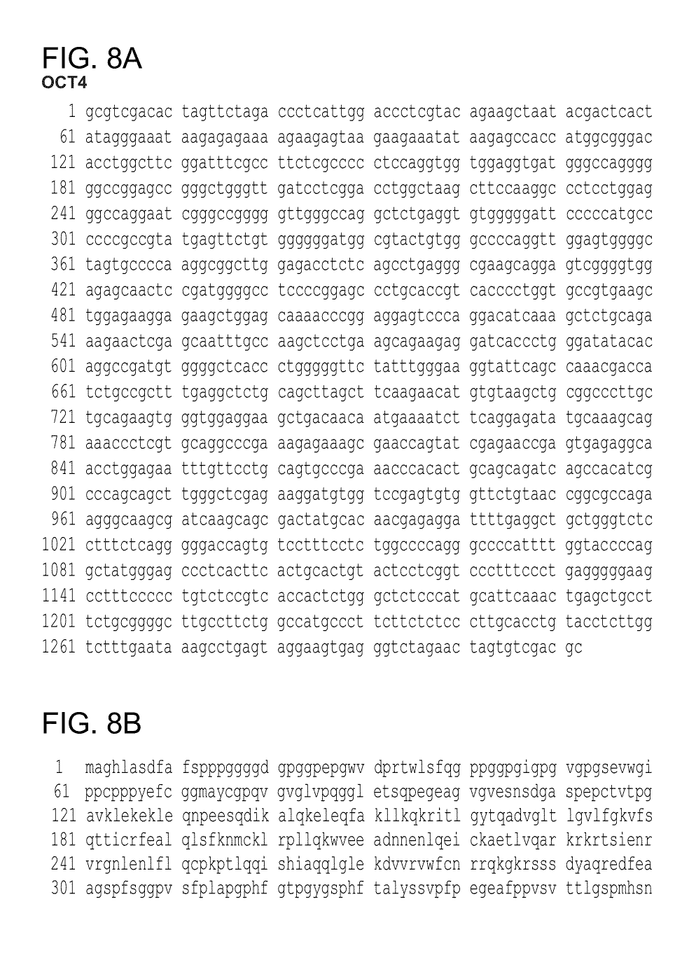

Preferably, “OCT4 protein” or simply “OCT4” relates to human OCT4 and preferably comprises an amino acid sequence encoded by the nucleic acid according to FIG. 8A, preferably the amino acid sequence according to FIG. 8B. One skilled in the art would understand that the cDNA sequence of OCT4 as described above would be equivalent to OCT4 mRNA, and can be used for the generation of RNA capable of expressing OCT4.

Sox2 is a member of the Sox (SRY-related HMG box) gene family that encode transcription factors with a single HMG DNA-binding domain. SOX2 has been found to control neural progenitor cells by inhibiting their ability to differentiate. The repression of the factor results in delamination from the ventricular zone, which is followed by an exit from the cell cycle. These cells also begin to lose their progenitor character through the loss of progenitor and early neuronal differentiation markers.

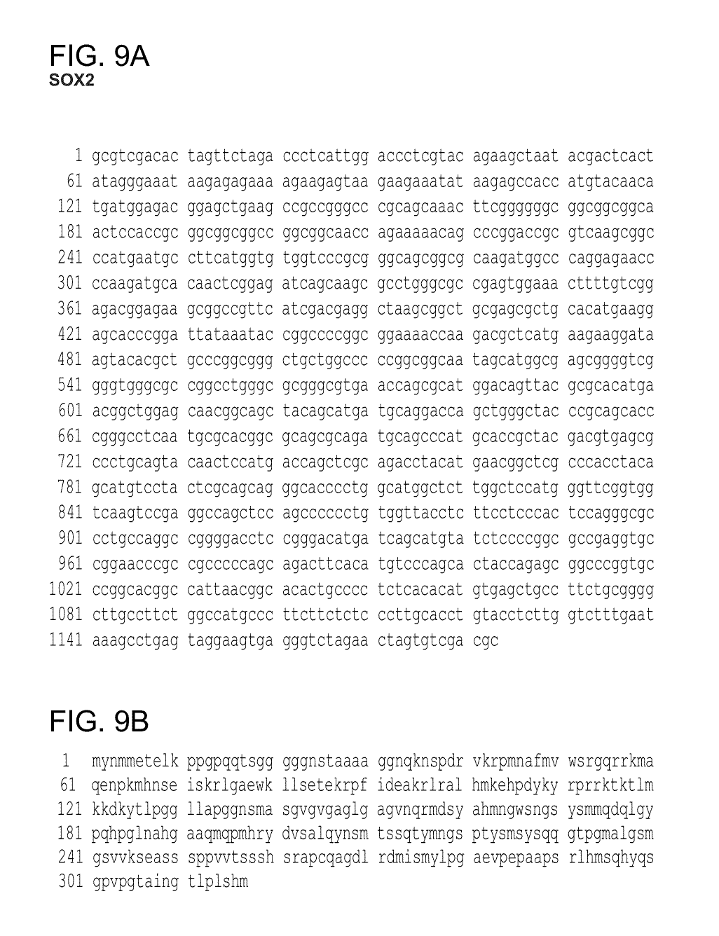

Preferably, “SOX2 protein” or simply “SOX2” relates to human SOX2 and preferably comprises an amino acid sequence encoded by the nucleic acid according to FIG. 9A, preferably the amino acid sequence according to FIG. 9B. One skilled in the art would understand that the cDNA sequence of SOX2 as described above would be equivalent to SOX2 mRNA, and can be used for the generation of RNA capable of expressing SOX2.

NANOG is a NK-2 type homeodomain gene, and has been proposed to play a key role in maintaining stem cell pluripotency presumably by regulating the expression of genes critical to embryonic stem cell renewal and differentiation. NANOG behaves as a transcription activator with two unusually strong activation domains embedded in its C terminus. Reduction of NANOG expression induces differentiation of embryonic stem cells.

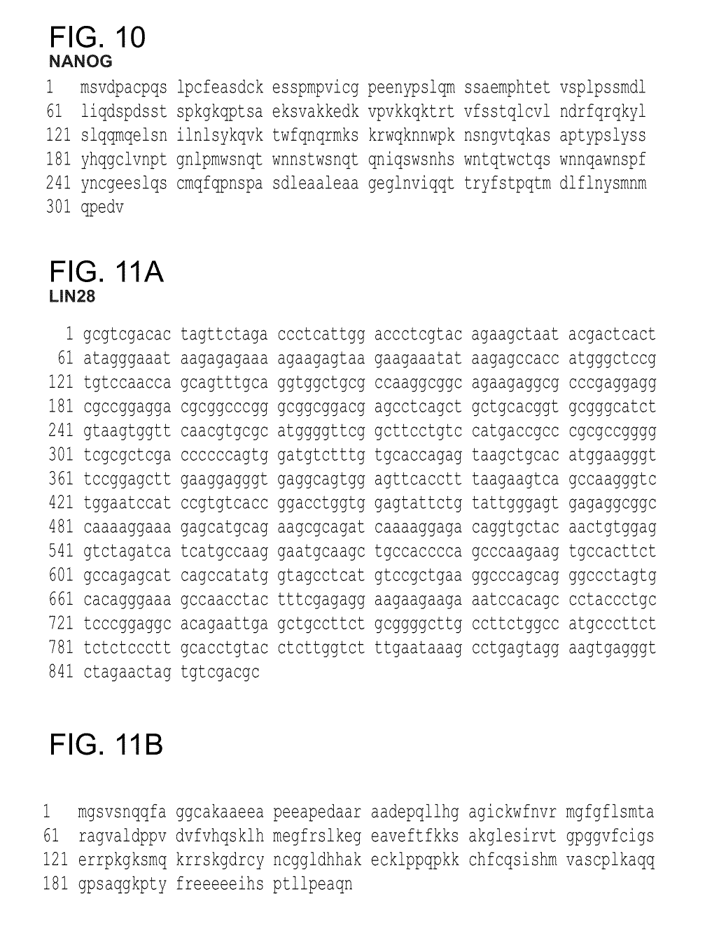

Preferably, “NANOG protein” or simply “NANOG” relates to human NANOG and preferably comprises an amino acid sequence encoded by the amino acid sequence according to FIG. 10. One skilled in the art would understand that the cDNA sequence of NANOG as described above would be equivalent to NANOG mRNA, and can be used for the generation of RNA capable of expressing NANOG.

LIN28 is a conserved cytoplasmic protein with an unusual pairing of RNA-binding motifs: a cold shock domain and a pair of retroviral-type CCHC zinc fingers. In mammals, it is abundant in diverse types of undifferentiated cells. In pluripotent mammalian cells, LIN28 is observed in RNase-sensitive complexes with Poly(A)-Binding Protein, and in polysomal fractions of sucrose gradients, suggesting it is associated with translating mRNAs.

Preferably, “LIN28 protein” or simply “LIN28” relates to human LIN28 and preferably comprises an amino acid sequence encoded by the nucleic acid according to FIG. 11A, preferably the amino acid sequence according to FIG. 11B. One skilled in the art would understand that the cDNA sequence of LIN28 as described above would be equivalent to LIN28 mRNA, and can be used for the generation of RNA capable of expressing LIN28.

Krueppel-like factor (KLF4) is a zinc-finger transcription factor, which is strongly expressed in postmitotic epithelial cells of different tissues, e.g. the colon, the stomach and the skin. KLF4 is essential for the terminal differentiation of these cells and involved in the cell cycle regulation.

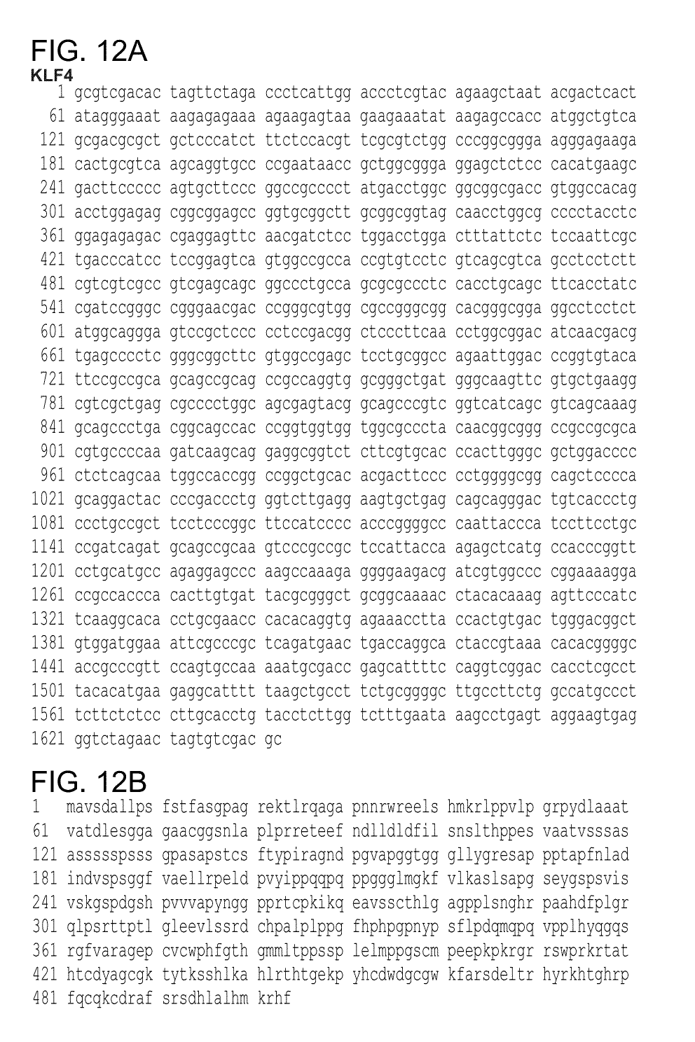

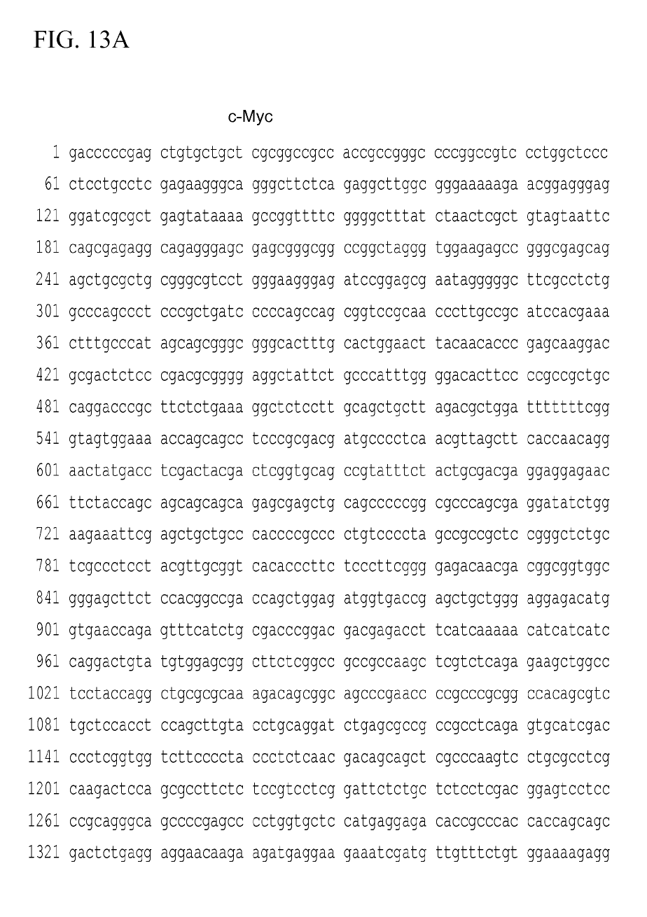

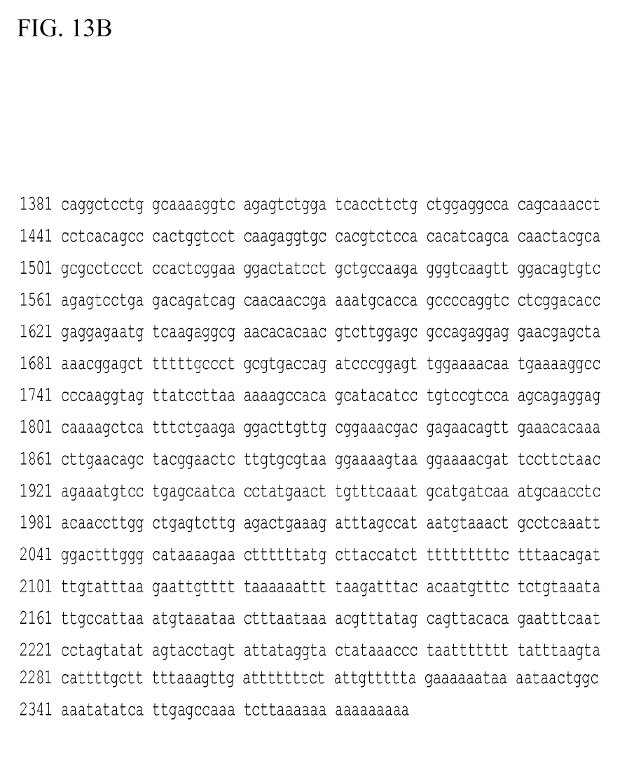

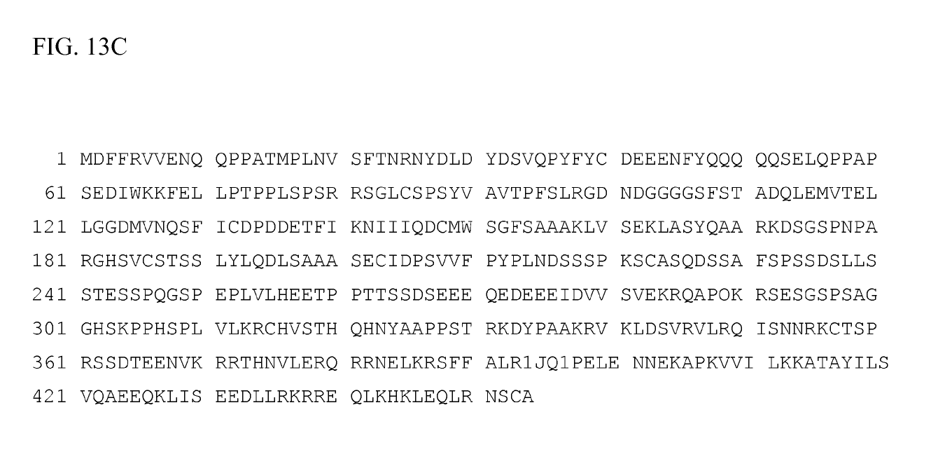

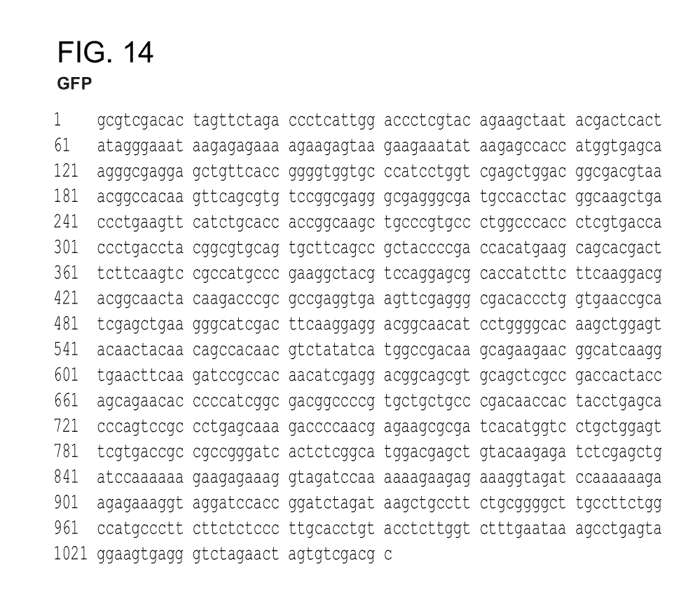

Preferably, “KLF4 protein” or simply “KLF4” relates to human KLF4 and preferably comprises an amino acid sequence encoded by the nucleic acid according to FIG. 12A, preferably the amino acid sequence according to FIG. 12B. One skilled in the art would understand that the cDNA sequence of KLF4 as described above would be equivalent to KLF4 mRNA, and can be used for the generation of RNA capable of expressing KLF4.

MYC (cMYC) is a protooncogene, which is overexpressed in a wide range of human cancers. When it is specifically-mutated, or overexpressed, it increases cell proliferation and functions as an oncogene. MYC gene encodes for a transcription factor that regulates expression of 15% of all genes through binding on Enhancer Box sequences (E-boxes) and recruiting histone acetyltransferases (HATs). MYC belongs to MYC family of transcription factors, which also includes N-MYC and L-MYC genes. MYC-family transcription factors contain the bHLH/LZ (basic Helix-Loop-Helix Leucine Zipper) domain.

Preferably, “cMYC protein” or simply “cMYC” relates to human cMYC and preferably comprises an amino acid sequence encoded by the nucleic acid according to FIGS. 13A-B, preferably the amino acid sequence according to FIG. 13C. One skilled in the art would understand that the cDNA sequence of cMYC as described above would be equivalent to cMYC mRNA, and can be used for the generation of RNA capable of expressing cMYC.

A reference herein to specific factors such as OCT4, SOX2, NANOG, LIN28, KLF4 or c-MYC or to specific sequences thereof is to be understood so as to also include all variants of these specific factors or the specific sequences thereof as described herein. In particular, it is to be understood so as to also include all splice variants, posttranslationally modified variants, conformations, isoforms and species homologs of these specific factors/sequences which are naturally expressed by cells.

A reprogramming factor or nuclear reprogramming factor useful according to the invention includes any of the reprogramming factors recited herein. A reprogramming factor useful according to the invention also includes a factor identified by the method of screening for reprogramming factors described in International Publication No. WO2005/80598 A1, incorporated by reference herein in its entirety. Those skilled in the art are able to screen a reprogramming factor for use in the method of the present invention by referring to the above publication. In addition, the reprogramming factor can also be confirmed by using a method in which appropriate modification or alteration has been made in the above screening method.

Examples of the combination reprogramming factors are disclosed in International Publication No, WO2007/069666 A1 and its family member U.S. patent application Ser. No. 12/213,035 and U.S. patent application Ser. No. 12/289,873, filed Nov. 6, 2008, entitled “Nuclear Reprogramming Factor and Induced Pluripotent Stem Cells” which are incorporated by reference herein in their entireties. Those skilled in the art are able to appropriately select a reprogramming factor that can be preferably used for the method of the present invention by referring to the above publication. In addition, other examples of the combinations of reprogramming factors are disclosed, for example, in Yu et al., Science 318:1917-20, 2007, incorporated by reference herein in its entirety. Accordingly, those skilled in the art are able to understand that any variety and combination of reprogramming factors can be used for the methods of the present invention, which combination is not disclosed in International Publication No. WO2007/069666 A1 or Yu et al., Science 318:1917-20, 2007. Reprogramming factors useful for the invention are identified by using the screening method of reprogramming factor described in International Publication No. WO2005/80598 A1.

The amino acid and nucleotide sequences of nuclear reprogramming factors usable alone or in combination in the present application, for example Oct3/4 Nanog, Lin28, Lin28b, ECAT1, ECAT2, ECAT3, ECAT5, ECAT7, ECAT8, ECAT9, ECAT10, ECAT15-1, ECAT15-2, Fthl17, Sal14, Rex1, Uff1, Tcl1, Stella, β-catenin, Stat3, Grb2, c-Myc, N-Myc, L-Myc, Sox1, Sox2, Sox3, Sox4, Sox11, Myb12, Klf1, Klf2, Klf4, and Klf5; and FoxD3, ZNF206, and Otx2 (which are also described in International Publication No. WO2008/118820), are available from GenBank (NCBI, USA). Accession numbers thereof regarding human, mouse or rat are described below:

Oct3/4 (human NM_{—}203289 or NM_{—}002701, mouse NM_{—}013633, rat NM_{—}001009178), Nanog (human NM_{—}024865, mouse NM_{—}028016, rat NM_{—}001100781), Lin28 (human NM_{—}024674, mouse NM_{—}145833, rat NM_{—}001109269), Lin28b (human NM_{—}001004317, mouse NM_{—}001031772), ECAT1 (human AB211062, mouse AB211060), ECAT2 (human NM_{—}001025290, mouse NM_{—}025274), ECAT3 (human NM_{—}152676, mouse NM_{—}015798), ECAT5 (human NM_{—}181532, mouse NM_{—}181548), ECAT7 (human NM_{—}013369, mouse NM_{—}019448), ECAT8 (human AB211063, mouse AB211061), ECAT9 (human NM_{—}020634, mouse NM_{—}008108), ECAT10 (human NM_{—}006942NM_{—}009235, mouse NM_{—}009235), ECAT15-1 (human NM_{—}018189, mouse NM_{—}028610), ECAT15-2 (human NM_{—}138815NM_{—}028615, mouse NM_{—}028615), Fthl17 (human NM_{—}031894, mouse NM_{—}031261), Sal14 (human NM_{—}020436, mouse NM_{—}175303), Rex1 (human NM_{—}174900, mouse NM_{—}609556), Uff1 (human NM_{—}003577, mouse NM_{—}009482), Tcl1 (human NM_{—}021966, mouse NM_{—}009337), Stella (human NM_{—}199286, mouse NM_{—}139218), β-catenin (human NM_{—}001904, mouse NM_{—}007614), Stat3 (human NM_{—}139276, mouse NM_{—}213659), Grb2 (human NM_{—}002086, mouse NM_{—}008163), FoxD3 (human NM_{—}012183, mouse NM_{—}010425), ZNF206 (human NM_{—}032805, mouse NM_{—}001033425), Myb12 (human NM_{—}002466, mouse NM_{—}008652), Otx2 (human NM_{—}172337, mouse NM_{—}144841), c-Myc (human NM_{—}002467, mouse NM_{—}010849), N-Myc (human NM_{—}005378, mouse NM_{—}008709), L-Myc (human NM_{—}005376, mouse NM_{—}008506), Sox1 (human NM_{—}005986 NM_{—}005986, NM_{—}009233, mouse NM_{—}005986, NM_{—}009233), Sox2 (human NM_{—}003106, mouse NM_{—}011443), Sox3 (human NM_{—}005634, mouse NM_{—}009237), Sox4 (human NM_{—}003107, mouse NM_{—}009238), Sox11 (human NM_{—}003108, mouse NM_{—}009234), Myb12 (human NM_{—}002466, mouse NM_{—}008652), Klf1 (human NM_{—}006563, mouse NM_{—}010635), Klf2 (human NM_{—}016270, mouse NM_{—}008452), Klf4 (human NM_{—}004235, mouse NM_{—}010637), and Klf5 (human NM_{—}001730, mouse NM-009769).

The reprogramming factors of the invention may further be combined with one or more gene product(s) of gene(s) selected from: Fbx15, Nanog, ERas, ECAT15-2, Tcl1, and β-catenin. Further, these factors may also be combined with one or more gene product(s) of gene(s) selected from: ECAT1, Esg1, Dnmt3L, ECAT8, Gdf3, Sox15, ECAT15-1, Fthl17, Sal14, Rex1, UTF1, Stella, Stat3, and Grb2, for example. However, gene products that can be included with the reprogramming factors of the present invention are not limited to the gene products of genes specifically described above. The nuclear reprogramming factors of the present invention can include other gene products which can function as a reprogramming factor, as well as one or more factors involving differentiation, development, or proliferation, and factors having other physiological activities. It should be understood that the aforementioned aspect may also be included within the scope of the present invention.

According to the present invention, the term “peptide” comprises oligo- and polypeptides and refers to substances comprising two or more, preferably 3 or more, preferably 4 or more, preferably 6 or more, preferably 8 or more, preferably 10 or more, preferably 13 or more, preferably 16 or more, preferably 21 or more and up to preferably 8, 10, 20, 30, 40 or 50, in particular 100 amino acids joined covalently by peptide bonds. The term “protein” refers to large peptides, preferably to peptides with more than 100 amino acid residues, but in general the terms “peptides” and “proteins” are synonyms and are used interchangeably herein.

Proteins and peptides described according to the invention may be isolated from biological samples such as tissue or cell homogenates and may also be expressed recombinantly in a multiplicity of pro- or eukaryotic expression systems.

For the purposes of the present invention, “variants” of a protein or peptide or of an amino acid sequence comprise amino acid insertion variants, amino acid deletion variants and/or amino acid substitution variants.

Amino acid insertion variants comprise amino- and/or carboxy-terminal fusions and also insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

“Conservative substitutions” may be made, for instance, on the basis of similarity in polarity, charge, solubility, hydrophobicity, hydrophilicity, and/or the amphipathic nature of the residues involved. Preferably the degree of similarity, preferably identity between a specific amino acid sequence described herein and an amino acid sequence which is a variant of said specific amino acid sequence will be at least 70%, preferably at least 80%, preferably at least 85%, even more preferably at least 90% or most preferably at least 95%, 96%, 97%, 98% or 99%. The degree of similarity or identity is given preferably for a region of at least about 20, at least about 40, at least about 60, at least about 80, at least about 100, at least about 120, at least about 140, at least about 160, at least about 200 or 250 amino acids. In preferred embodiments, the degree of similarity or identity is given for the entire length of the reference amino acid sequence.

According to the invention, a variant of a protein or peptide preferably has a functional property of the protein or peptide from which it has been derived. Such functional properties are described above for OCT4, SOX2, NANOG, LIN28, KLF4 and c-MYC, respectively. Preferably, a variant of a protein or peptide has the same property in reprogramming an animal differentiated cell as the protein or peptide from which it has been derived. Preferably, the variant induces or enhances reprogramming of an animal differentiated cell.

miRNAs

The invention provides for methods of producing a pluripotent stem cell wherein one or more miRNA(s) is introduced into a target cell in combination with mRNA.

One microRNA cluster, designated the miR-290 cluster, constitutes over 70% of the entire miRNA population in mouse ES cells (Marson, A. et al. Connecting microRNA genes to the core transcriptional regulatory circuitry of embryonic stem cells Cell 134:521-533 (2008)). Expression of the miR-290 duster is rapidly down-regulated upon ES cell differentiation (See, e.g., Houbaviy, H. B., Murray, M. F. & Sharp, P. A. Embryonic stem cell-specific MicroRNAs Dev Cell 5:351-358 (2003)). A subset of the miR-290 duster, called the embryonic stem cell cycle (ESCC) regulating miRNAs, enhances the unique stem cell cycle and includes miR-291-3p, miR-294, and miR-295, as well as the human homologues hsa-mir-302a, hsa-miR-302b, hsa-miR-302c, hsa-miR-302d, hsa-miR-371-5p, hsa-miR-372, hsa-miR-373. (See, e.g., Wang, Y. et al. Embryonic stem cell-specific microRNAs regulate the G1-S transition and promote rapid proliferation Nat Genet 40:1478-1483 (2008)). This subset includes miR-291-3p, miR-294, and miR-295 and their homologues.

Removal of genes required for maturation of all miRNAs has shown that miRNAs play essential roles in the proliferation and differentiation of Embryonic Stem Cells (ESCs)(Wang, Y. et al., Nat Genet 39:380-5 (2007); Kanellopoulou, C. et al. Genes Dev 19:489-501 (2005); Murchison, E. P. et al., Proc Natl Acad Sci USA 102:12135-40 (2005)). For example, the loss of the RNA binding protein DGCR8, which is required for the production of all canonical miRNAs, results in a cell cycle defect and an inability to silence the self-renewal program of ESCs when they are placed in differentiation-inducing conditions (Wang, Y. et al., Nat Genet 39:380-5 (2007). The introduction of individual members of a family of miRNAs, the ESCC miRNAs, into Dgcr8−/−ESCs can rescue the cell cycle defect (Wang, Y. et al., Nat Genet, 40:1478-1483 (2008)).

According to the methods of the invention, miRNA comprises one or more miRNA(s) included in the RNA sequences specified by the registration names of the miRBase database or the accession numbers shown in the tables below or the sequences or combination of sequences shown in the tables below or any possible combination of the sequences shown below. In the registration names, the symbols “hsa” and “mmu” represent Homo sapiens and Mus musculus, respectively.

The invention provides for an miRNA that is 18-25 nucleotides, for example, 20-25 nucleotides, 21-23 nucleotides and 19-23 nucleotides. Such miRNAs can be induced from precursor RNAs including pri-miRNAs (i.e., transcription products from genomic DNAs) and pre-miRNAs (i.e., processed products from pri-miRNAs).

The present invention provides methods comprising the use of miRNA that provide for a higher reprogramming efficiency in the presence of the miRNA than in the absence thereof, for preparation of induced pluripotent stem cells. For example, the presence of an added miRNA supports the production of an induced pluripotent stem cell as compared to in the absence of the miRNA.

Further, when reprogramming is performed on the same number of somatic cells in the presence of a reprogramming factor containing the same components in the same concentrations with and without addition of miRNA, increased efficiency can be observed wherein a greater number of induced pluripotent stem cells are generated in the sample which comprises miRNA as compared to the sample that does not comprise miRNA.

Regarding miRNA useful according to the invention, its classification and in vivo functions are described in Jikken Igaku (Experimental Medicine), 24, pp. 814-819, 2006; microRNA Jikken Purotokoru (microRNA Experimental Protocol), pp. 20-35, 2008, YODOSHA CO., LTD. At present, a database storing data relating to about 1,000 miRNA sequences is available (for example, miRBase, Griffiths-Jones et al. Nucleic Acids Research 36:D154-D158, 2008 (published online Nov. 8, 2007), see also http://microma.sanger.ac.uk/sequences/index.shtml [online]), and it is possible for those skilled in the art to obtain any miRNA data therefrom, and to readily extract an miRNA that is expressed in embryonic stem cells at a higher level than in somatic cells. In addition, it is also possible to readily identify miRNA expressed in embryonic stem cells at a higher level than in somatic cells by confirming the difference in miRNA expression between embryonic stem cells and somatic cells by methods including but not limited to miRNA microarray and real-time PCR analyses.

It is preferable to use miRNA derived from the same animal species as the target animal whose somatic cells are to be reprogrammed. miRNA useful according to the invention includes wild type miRNA as well as miRNAs in which one to several nucleotides (for example 1 to 6 nucleotides, preferably 1 to 4 nucleotides, more preferably 1 to 3 nucleotides, yet more preferably 1 or 2 nucleotides, and most preferably 1 nucleotide) are substituted, inserted, and/or deleted, and which are capable of exerting equivalent functions to those of the wild type miRNA in vivo. For example, the miRNA of the present invention includes miRNAs in which one to several nucleotides are substituted, inserted, and/or deleted, and which increase the efficiency of iPS cell production. The miRNA of the present invention also includes miRNAs in which one to several nucleotides are substituted, inserted, and/or deleted, and which improve the efficiency of nuclear reprogramming. The miRNA of the present invention also includes miRNAs in which one to several nucleotides are substituted, inserted, and/or deleted, and which regulate DNA methylation. The present invention also includes such miRNAs wherein the DNA methylation is down-regulated. The present invention also includes such miRNAs wherein the DNA methylation is de novo DNA methylation.

According to the methods of the present invention, miRNAs that have been confirmed to improve the reprogramming efficiency in the above manner can be used either alone or in combinations of two or more types. In addition, a plurality of miRNAs forming a duster may also be used. For example, hsa-miR-302-367 which is available as a miRNA cluster, or individual miRNAs from the hsa-miR-302-367 duster, and the like may be used. Among these RNA sequences, some RNA sequences may include a plurality of miRNAs within one sequence. Use of such an RNA sequence may achieve efficient production of iPS cells. Further, an RNA sequence including a plurality of miRNAs within one sequence and one or more other RNA sequence(s) including one or more miRNA(s) can also be used in combination. In the invention, preferably, the miRNAs are one or two or more miRNAs contained in one or two or more RNAs selected from RNAs represented in the tables presented below.

An miRNA is non-coding RNA which is not translated into a protein. miRNA is first transcribed as pri-miRNA from a corresponding gene. A pri-miRNA generates pre-miRNA having a characteristic hairpin structure of about 60 to about 120 nucleotides or more, for example about 70 nucleotides, and this pre-miRNA is further processed into mature miRNA, which is mediated by Dicer. In the present invention, not only mature miRNA but also precursor RNA thereof (i.e., pri-miRNA or pre-miRNA), or a vector comprising DNA encoding the miRNA or precursor RNA, can be used as long as the effect of the present invention is not impaired. In addition, miRNA for use in the present invention may be either natural type or non-natural type. Thus, any small RNA or RNA precursor may be used as long as the effect of the present invention is not impaired.

The production method of miRNA for use in the present invention is not specifically limited, although the production can be achieved, for example, by a chemical synthetic method or a method using genetic recombination technique. When the production is carried out by a method using genetic recombination technique, miRNA for use in the present invention can, for example, be produced through a transcription reaction with use of a DNA template and a RNA polymerase obtained by means of gene recombination. Examples of usable RNA polymerase include a T7 RNA polymerase, a T3 RNA polymerase, and a SP6 RNA polymerase.

Alternatively, a recombinant vector capable of expressing miRNA can be produced by insertion of miRNA-encoding DNA or precursor RNA (pri-miRNA or pre-miRNA)-encoding DNA into an appropriate vector under the regulation of expression control sequences (promoter and enhancer sequences and the like).

The type of vector used herein is not specifically limited, although DNA vectors are preferred. Examples thereof can include plasmid vectors. In addition, as to the above plasmids, mammalian expression plasmids well known to those skilled in the art can be employed.

The invention also provides for methods of producing pluripotent stem cells using miRNA mimics, as defined herein, in combination with mRNA. The invention also provides for pluripotent stem cells comprising at least one miRNA mimic and at least one mRNA.

An miRNA useful for the methods of the invention includes any miRNA known to be involved in pluripotency of a cell or the mesenchymal to epithelial transition. miRNA useful according to the invention include but are not limited to the following:

TABLE 1
Sequences of miRNA used to enhance cellular
reprogramming

| miRNA | Sequence |
|---|---|
| hsa-mir-302a | CCACCACUUAAACGUGGAUGUACUUGCUUUGAAA |
| | CUAAAGAAGUAAGUGCUUCCAUGUUUUGGUGAUG |
| | G or UAAGUGCUUCCAUGUUUUGGUGA |
| hsa-mir-302b | GCUCCCUUCAACUUUAACAUGGAAGUGCUUUCUG |
| | UGACUUUAAAAGUAAGUGCUUCCAUGUUUUAGUA |
| | GGAGU or UAAGUGCUUCCAUGUUUUAGUAG |
| hsa-mir-302c | CCUUUGCUUUAACAUGGGGGUACCUGCUGUGUGA |
| | AACAAAAGUAAGUGCUUCCAUGUUUCAGUGGAGG |
| | or UAAGUGCUUCCAUGUUUCAGUGG |
| hsa-mir-302d | CCUCUACUUUAACAUGGAGGCACUUGCUGUGACA |
| | UGACAAAAAUAAGUGCUUCCAUGUUUGAGUGUGG |
| | or UAAGUGCUUCCAUGUUUGAGUGU |
| hsa-mir-367 | CCAUUACUGUUGCUAAUAUGCAACUCUGUUGAAU |
| | AUAAAUUGGAAUUGCACUUUAGCAAUGGUGAUGG |
| | or AAUUGCACUUUAGCAAUGGUGA |
| hsa-mir-200c | CCCUCGUCUUACCCAGCAGUGUUUGGGUGCGGUU |
| | GGGAGUCUCUAAUACUGCCGGGUAAUGAUGGAGG |
| hsa-mir-369-3p | AAUAAUACAUGGUUGAUCUUU |
| hsa-mir-369-5p | AGAUCGACCGUGUUAUAUUCGC |

In certain embodiments, combinations of miRNAs are introduced into a somatic cell to facilitate production of a pluripotent stem cell (see for example Table 2).

TABLE 2
miRNA cocktails used in combination with mRNA reprogramming

| Cluster A | Cluster B |
|---|---|
| 302a, 302b, 302c, 302d, | 302a, 302b, 302c, 302d, |
| 367 | 200c, 369-3p, 369-5p |

TABLE 3
Sequences of miRNA used to enhance cellular reprogramming

| miRNA | miR Base Accession Number |
|---|---|
| mmu-miR-150 | MI0000172 |
| mmu-miR-182 | MI0000224 |
| mmu-miR-126 | MI0000153 |
| 4 mmu-miR-290-295 | |
| cluster | |
| mmu-miR-290 | MI0000388 |
| (mmu-miR-290-5p/290-3p) | |
| mmu-miR-291a | MI0000389 |
| (mmu-miR-291a-5p/291a- | |
| 3p) | |
| mmu-miR-292 | MI0000390 |
| (mmu-miR-292-5p/292-3p) | |
| mmu-miR-294 | MI0000392 |
| (mmu-miR-294/294*) | |
| mmu-miR-295 | MI0000393 |
| (mmu-miR-295/295*) | |
| mmu-miR-17-92 cluster | |
| mmu-miR-323 | MI0000592 |
| mmu-miR-130b | MI0000408 |
| mmu-miR-7a-1 | MI0000728 |
| 14 mmu-miR-7a-2 | MI0000729 |
| mmu-miR-205 | MI0000248 |
| mmu-miR-200a | MI0000554 |
| 17 mmu-miR-200c | MI0000694 |
| mmu-miR-mix | |

*indicates star form of miRNA.

TABLE 4
Sequences of miRNA used to enhance cellular reprogramming

| miRNA | miR Base Accession Number |
|---|---|
| hsa-miR-371 | MI0000779 |
| (hsa-miR-371-5p/371-3p) | |
| hsa-miR-372 | MI0000780 |
| hsa-miR-373 | MI0000781 |
| (hsa-miR-373/373*) | |
| hsa-miR-371-373 cluster | |
| hsa-miR-93 | MI0000095 |
| (hsa-miR-93/93*) | |
| hsa-miR-302a | MI0000738 |
| (hsa-miR-302a/302a*) | |
| hsa-miR-302b | MI0000772 |
| (hsa-miR-302b/302b*) | |
| hsa-miR-302c | MI0000773 |
| (hsa-miR-302c/302c*) | |
| hsa-miR-302d | MI0000774 |
| (hsa-miR-302d/302d*) | |
| hsa-miR-367 | MI0000775 |
| (hsa-miR-367/367*) | |
| hsa-miR-302-367 cluster | |
| hsa-miR-520a | MI0003149 |
| (hsa-miR-520a-5p/520a-3p) | |
| hsa-miR-520b | MI0003155 |
| hsa-miR-520c | MI0003158 |
| (hsa-miR-520c-5p/520c-3p) | |
| hsa-miR-520d | MI0003164 |
| (hsa-miR-520d-5p/520d-3p) | MI0003143 |
| hsa-miR-520e | |
| mmu-miR-290-295 cluster | |
| mmu-miR-290 | MI0000388 |
| (mmu-miR-290-5p/290-3p) | |
| mmu-miR-291a | MI0000389 |
| (mmu-miR-291a-5p/291a-3p) | |
| mmu-miR-292 | MI0000390 |
| (mmu-miR-292-5p/292-3p) | |
| mmu-miR-293 | MI0000391 |
| (mmu-miR-293/293*) | |
| mmu-miR-294 | MI0000392 |
| (mmu-miR-294/294*) | |
| mmu-miR-295 | MI0000393 |
| (mmu-miR-295/295*) | |

TABLE 5

| microRNA mimic | sequence |
|---|---|
| mmu-miR-302b | UAAGUGCUUCCAUGUUUUAGUAG |
| mmu-miR-302 | UAAGUGCUUCCAUGUUUUGGUGA |
| mmu-miR-495 | AAACAAACAUGGUGCACUUCUU |
| mmu-miR-26a | UUCAAGUAAUCCAGGAUAGGCU |
| mmu-miR-19a* | UAGUUUUGCAUAGUUGCACUAC |
| mmu-miR-302d | UAAGUGCUUCCAUGUUUGAGUGU |
| mmu-miR-10b | UACCCUGUAGAACCGAAUUUGUG |
| mmu-miR-294 | AAAGUGCUUCCCUUUUGUGUGU |
| mmu-miR-302c | AAGUGCUUCCAUGUUUCAGUGG |
| mmu-miR-183* | GUGAAUUACCGAAGGGCCAUAA |
| mmu-miR-200a | UAACACUGUCUGGUAACGAUGU |
| mmu-miR34c* | AAUCACUAACCACACAGCCAGG |
| mmu-miR-293 | AGUGCCGCAGAGUUUGUAGUGU |
| mmu-miR-181b | AACAUUCAUUGCUGUCGGUGGGU |
| mmu-miR-151 | CUAGACUGAGGCUCCUUGAGG |
| mmu-miR-680 | GGGCAUCUGCUGACAUGGGGG |
| mmu-miR-295 | AAAGUGCUACUACUUUUGAGUCU |
| mmu-miR-880 | UACUCCAUCCUCUCUGAGUAGA |
| mmu-miR-93 | CAAAGUGCUGUUCGUGCAGGUAG |
| mmu-miR-455-5p | UAUGUGCCUUUGGACUACAUCG |
| mmu-miR-144 | UACAGUAUAGAUGAUGUACU |
| mmu-miR-467d | UAAGUGCGCGCAUGUAUAUGCG |
| mmu-miR-484 | UCAGGCUCAGUCCCCUCCCGAU |
| mmu-miR-205 | UCCUUCAUUCCACCGGAGUCUG |
| mmu-miR-582-5p | UACAGUUGUUCAACCAGUUACU |
| mmu-miR-290-3p | AAAGUGCCGCCUAGUUUUAAGCCC or |
| | AAAGUGCCGCCUAGUUUUAAGCC |
| mmu-miR-138* | CGGCUACUUCACAACACCAGGG |
| mmu-miR-181d | AACAUUCAUUGUUGUCGGUGGGU |
| mmu-miR-324-3p | CCACUGCCCCAGGUGCUGCU |
| mmu-miR-877* | UGUCCUCUUCUCCCUCCUCCCA |
| mmu-miR-23a | AUCACAUUGCCAGGGAUUUCC |
| mmu-miR-379 | UGGUAGACUAUGGAACGUAGG |
| mmu-miR-673 | CUCACAGCUCUGGUCCUUGGAG |
| mmu-miR-876-5p | UGGAUUUCUCUGUGAAUCACUA |
| mmu-miR-291-3p | AAAGUGCUUCCACUUUGUGUGC |
| mmu-miR-30d | UGUAAACAUCCCCGACUGGAAG |
| mmu-miR-421 | AUCAACAGACAUUAAUUGGGCGC |
| mmu-miR-879* | GCUUAUGGCUUCAAGCUUUCGG |
| mmu-miR-542-3p | UGUGACAGAUUGAUAACUGAAA |
| mmu-miR-124* | CGUGUUCACAGCGGACCUUGAU |
| mmu-miR-363 | AAUUGCACGGUAUCCAUCUGUA |
| mmu-miR-871 | UAUUCAGAUUAGUGCCAGUCAUG |
| mmu-miR-19a | UGUGCAAAUCUAUGCAAAACUGA |
| mmu-miR-16* | CCAGUAUUGACUGUGCUGCUGA |
| mmu-miR-873 | GCAGGAACUUGUGAGUCUCCU |
| mmu-miR-199b | CCCAGUGUUUAGACUACCUGUUC |
| mmu-miR-106a | CAAAGUGCUAACAGUGCAGGUAG |
| mmu-miR-181b | AACAUUCAUUGCUGUCGGUGGGU |
| mmu-miR-200a* | CAUCUUACCGGACAGUGCUGGA |
| mmu-miR-431* | CAGGUCGUCUUGCAGGGCUUCU |
| mmu-miR-689 | CGUCCCCGCUCGGCGGGGUCC |
| mmu-miR-721 | CAGUGCAAUUAAAAGGGGGAA |

TABLE 6

| microRNA mimic | sequence |
|---|---|
| mmu-miR-744* | CUGUUGCCACUAACCUCAACCU |
| mmu-miR-423-5p | UGAGGGGCAGAGAGCGAGACUUU |
| mmu-miR-669c | AUAGUUGUGUGUGGAUGUGUGU |
| mmu-let-7c | UGAGGUAGUAGGUUGUAUGGUU |
| mmu-miR-466h | UGUGUGCAUGUGCUUGUGUGUA |
| mmu-miR-654-3p | UAUGUCUGCUGACCAUCACCUU |
| mmu-miR-470* | AACCAGUACCUUUCUGAGAAGA |
| mmu-miR-24 | UGGCUCAGUUCAGCAGGAACAG |
| mmu-miR-182 | UUUGGCAAUGGUAGAACUCACACCG |
| mmu-miR-335 | UCAAGAGCAAUAACGAAAAAUGU |
| mmu-miR-181c | AACAUUCAACCUGUCGGUGAGU |
| mmu-miR-330 | GCAAAGCACAGGGCCUGCAGAGA |
| mmu-miR-134 | UGUGACUGGUUGACCAGAGGGG |
| mmu-miR-675-3p | CUGUAUGCCCUAACCGCUCAGU |
| mmu-miR-218 | UUGUGCUUGAUCUAACCAUGU |
| mmu-let-7f | UGAGGUAGUAGGUUGUAUGGUU |
| mmu-miR-491 | AGUGGGGAACCCUUCCAUGAGG |
| mmu-miR-466g | AUACAGACACAUGCACACACA |
| mmu-miR-465c-3p | GAUCAGGGCCUUUCUAAGUAGA |
| mmu-miR-202 | AGAGGUAUAGCGCAUGGGAAGA |
| mmu-miR-681 | CAGCCUCGCUGGCAGGCAGCU |
| mmu-miR-877 | GUAGAGGAGAUGGCGCAGGG |
| mmu-miR-875-5p | UAUACCUCAGUUUUAUCAGGUG |
| mmu-miR-712 | CUCCUUCACCCGGGCGGUACC |
| mmu-miR-297 | AUGUAUGUGUGCAUGUGCAUGU |
| mmu-let-7d | AGAGGUAGUAGGUUGCAUAGUU |
| mmu-miR-142-3p | UGUAGUGUUUCCUACUUUAUGGA |
| mmu-miR-328 | CUGGCCCUCUCUGCCCUUCCGU |
| mmu-miR-485-5p | AGAGGCUGGCCGUGAUGAAUUC |
| mmu-miR-122a | UGGAGUGUGACAAUGGUGUUUG |
| mmu-miR-877* | UGUCCUCUUCUCCCUCCUCCCA |
| mmu-miR-135a | UAUGGCUUUUUAUUCCUAUGUGA |
| mmu-miR-674-3p | CACAGCUCCCAUCUCAGAACAA |
| mmu-miR-497 | CAGCAGCACACUGUGGUUUGUA |
| mmu-miR-7b | UGGAAGACUUGUGAUUUUGUUGU |
| mmu-miR-30b* | CUGGGAUGUGGAUGUUUACGUC |
| mmu-miR-34b | AGGCAGUGUAAUUAGCUGAUUGU |
| mmu-miR-466e-5p | GAUGUGUGUGUACAUGUACAUA |
| mmu-miR-193b | AACUGGCCCACAAAGUCCCGCU |
| mmu-miR-883a-5p | UGCUGAGAGAAGUAGCAGUUAC |
| mmu-let-7i* | CUGCGCAAGCUACUGCCUUGCU |
| mmu-miR-342 | UCUCACACAGAAAUCGCACCCGU |
| mmu-miR-140* | UACCACAGGGUAGAACCACGG |
| mmu-miR-24-2* | GUGCCUACUGAGCUGAAACAGU |
| mmu-miR-195 | UAGCAGCACAGAAAUAUUGGC |
| mmu-miR-297a | AUGUAUGUGUGCAUGUGCAUGU |
| mmu-miR-344 | UGAUCUAGCCAAAGCCUGACUGU |
| mmu-miR-18 | UAAGGUGCAUCUAGUGCAGAUAG |
| mmu-miR-93* | ACUGCUGAGCUAGCACUUCCCG |
| mmu-miR-297 | AUGUAUGUGUGCAUGUGCAUGU |
| mmu-miR-16 | UAGCAGCACGUAAAUAUUGGCG |
| mmu-miR-380-5p | AUGGUUGACCAUAGAACAUGCG |
| mmu-miR-672 | UGAGGUUGGUGUACUGUGUGUGA |
| mmu-miR-431 | UGUCUUGCAGGCCGUCAUGCA |
| mmu-miR-715 | CUCCGUGCACACCCCCGCGUG |
| mmu-miR-669a | AGUUGUGUGUGCAUGUUCAUGU |
| mmu-miR-103 | AGCAGCAUUGUACAGGGCUAUGA |
| mmu-miR-124* | CGUGUUCACAGCGGACCUUGAU |
| mmu-miR-15b | UAGCAGCACAUCAUGGUUUACA |
| mmu-miR-450b* | AUUGGGAACAUUUUGCAUGCAU |
| mmu-miR-882 | AGGAGAGAGUUAGCGCAUUAGU |
| mmu-miR-686 | AUUGCUUCCCAGACGGUGAAGA |
| mmu-miR-222 | AGCUACAUCUGGCUACUGGGU |
| mmu-miR-684 | AGUUUUCCCUUCAAGUCAA |
| mmu-miR-450b | UUUUGCAGUAUGUUCCUGAAUA |
| mmu-miR-582-3p | CCUGUUGAACAACUGAACCCAA |
| mmu-miR-135b | UAUGGCUUUUCAUUCCUAUGUGA |
| mmu-miR-493 | UGAAGGUCCUACUGUGUGCCAGG |
| mmu-miR-546 | AUGGUGGCACGGAGUC |
| mmu-miR-708 | AAGGAGCUUACAAUCUAGCUGGG |
| mmu-miR-433-3p | AUCAUGAUGGGCUCCUCGGUGU |
| mmu-miR-494 | UGAAACAUACACGGGAAACCUC |
| mmu-miR-203 | GUGAAAUGUUUAGGACCACUAG |
| mmu-miR-9 | UCUUUGGUUAUCUAGCUGUAUGA |
| mmu-miR-574-5p | UGAGUGUGUGUGUGUGAGUGUGU |
| mmu-miR-376c | AACAUAGAGGAAAUUUCACGU |
| mmu-miR-433-5p | UACGGUGAGCCUGUCAUUAUUC |
| mmu-miR-181a-2* | ACCGACCGUUGACUGUACCUUG |
| mmu-miR-218-2* | CAUGGUUCUGUCAAGCACCGCG |
| mmu-miR-196a | UAGGUAGUUUCAUGUUGUUGGG |
| mmu-miR-542-5p | CUCGGGGAUCAUCAUGUCACGA |
| mmu-miR-7 | UGGAAGACUAGUGAUUUUGUUGU |
| mmu-miR-743b-5p | UGUUCAGACUGGUGUCCAUCA |
| mmu-miR-377 | AUCACACAAAGGCAACUUUUGU |
| mmu-miR-683 | CCUGCUGUAAGCUGUGUCCUC |
| mmu-miR-675-5p | UGGUGCGGAAAGGGCCCACAGU |
| mmu-miR-598 | UACGUCAUCGUCGUCAUCGUUA |
| mmu-miR15b* | CGAAUCAUUAUUUGCUGCUCUA |
| mmu-miR-9 | UCUUUGGUUAUCUAGCUGUAUGA |
| mmu-miR-450a-3p | AUUGGGGAUGCUUUGCAUUCAU |
| mmu-miR-449b | AGGCAGUGUUGUUAGCUGGC |
| mmu-miR-707 | CAGUCAUGCCGCUUGCCUACG |
| mmu-miR-335-3p | UUUUUCAUUAUUGCUCCUGACC |
| mmu-miR-147 | GUGUGCGGAAAUGCUUCUGCUA |
| mmu-miR-466c-5p | GAUGUGUGUGUGCAUGUACAUA |
| mmu-miR-16 | UAGCAGCACGUAAAUAUUGGCG |
| mmu-miR-127 | UCGGAUCCGUCUGAGCUUGGCU |
| mmu-miR-673-3p | UCCGGGGCUGAGUUCUGUGCACC |
| mmu-miR-466b-5p | GAUGUGUGUGUACAUGUACAUG |
| mmu-miR-27a* | AGGGCUUAGCUGCUUGUGAGCA |
| mmu-miR-1 | UGGAAUGUAAAGAAGUAUGUAU |
| mmu-miR-201 | UACUCAGUAAGGCAUUGUUCUU |
| mmu-miR-376b | AUCAUAGAGGAACAUCCACUU |
| mmu-miR-187 | UCGUGUCUUGUGUUGCAGCCGG |
| mmu-miR-299 | UGGUUUACCGUCCCACAUACAU |
| mmu-miR-299 | UAUGUGGGACGGUAAACCGCUU |
| mmu-miR-574-3p | CACGCUCAUGCACACACCCACA |
| mmu-miR-193* | UGGGUCUUUGCGGGCAAGAUGA |
| mmu-miR-679 | GGACUGUGAGGUGACUCUUGGU |
| mmu-miR-540-5p | CAAGGGUCACCCUCUGACUCUGU |
| mmu-miR-466a-5p | UAUGUGUGUGUACAUGUACAUA |
| mmu-miR-470 | UUCUUGGACUGGCACUGGUGAGU |
| mmu-miR-1224 | GUGAGGACUGGGGAGGUGGAG |
| mmu-miR-191 | CAACGGAAUCCCAAAAGCAGCUG |
| hsa-miR-199b-5p | CCCAGUGUUUAGACUAUCUGUUC |
| hsa-let-7a | UGAGGUAGUAGGUUGUAUAGUU |
| hsa-let-7b | UGAGGUAGUAGGUUGUGUGGUU |
| hsa-let-7c | UGAGGUAGUAGGUUGUAUGGUU |
| hsa-let-7d | AGAGGUAGUAGGUUGCAUAGUU |
| hsa-let-7e | UGAGGUAGGAGGUUGUAUAGUU |
| hsa-let-7f | UGAGGUAGUAGAUUGUAUAGUU |
| hsa-let-7g | UGAGGUAGUAGUUUGUACAGUU |
| hsa-let-7i | UGAGGUAGUAGUUUGUGCUGUU |
| hsa-miR-100 | AACCCGUAGAUCCGAACUUGUG |
| hsa-miR-100 | AACCCGUAGAUCCGAACUUGUG |
| hsa-miR-122 | UGGAGUGUGACAAUGGUGUUUG |
| hsa-miR-127-3p | UCGGAUCCGUCUGAGCUUGGCU |
| hsa-miR-128 | UCACAGUGAACCGGUCUCUUU |
| hsa-miR-129-5p | CUUUUUGCGGUCUGGGCUUGC |
| hsa-miR-134 | UGUGACUGGUUGACCAGAGGGG |
| hsa-miR-140-5p | CAGUGGUUUUACCCUAUGGUAG |
| hsa-miR-145 | GUCCAGUUUUCCCAGGAAUCCCU |
| hsa-miR-149 | UCUGGCUCCGUGUCUUCACUCCC |
| hsa-miR-18a | UAAGGUGCAUCUAGUGCAGAUAG |
| hsa-miR-18b | UAAGGUGCAUCUAGUGCAGUUAG or |
| | UAAGGUGCAUCUAGUGCUGUUA |
| hsa-miR-193a-3p | AACUGGCCUACAAAGUCCCAGU |
| hsa-miR-199a-5p | CCCAGUGUUCAGACUACCUGUUC |
| hsa-miR-216a | UAAUCUCAGCUGGCAACUGUGA |
| hsa-miR-216b | AAAUCUCUGCAGGCAAAUGUGA |
| hsa-miR-218 | UUGUGCUUGAUCUAACCAUGU |
| hsa-miR-26a | UUCAAGUAAUCCAGGAUAGGCU |
| hsa-miR-31 | AGGCAAGAUGCUGGCAUAGCU |
| hsa-miR-345 | GCUGACUCCUAGUCCAGGGCUC |
| hsa-miR-34c-5p | AGGCAGUGUAGUUAGCUGAUUGC |
| hsa-miR-362-5p | AAUCCUUGGAACCUAGGUGUGAGU |
| hsa-miR-378 | ACUGGACUUGGAGUCAGAAGG |
| hsa-miR-384 | AUUCCUAGAAAUUGUUCAUA |
| hsa-miR-409-3p | GAAUGUUGCUCGGUGAACCCCU |
| hsa-miR-450a | UUUUGCGAUGUGUUCCUAAUAU |
| hsa-miR-450b-5p | UUUUGCAAUAUGUUCCUGAAUA |
| hsa-miR-452 | AACUGUUUGCAGAGGAAACUGA |
| hsa-miR-98 | UGAGGUAGUAAGUUGUAUUGUU |
| hsa-miR-99a | AACCCGUAGAUCCGAUCUUGUG |
| hsa-miR-99b | CACCCGUAGAACCGACCUUGCG |
| mmu-let-7a | UGAGGUAGUAGGUUGUAUAGUU |
| mmu-let-7b | UGAGGUAGUAGGUUGUGUGGUU |
| mmu-let-7c | UGAGGUAGUAGGUUGUAUGGUU |
| mmu-let-7d | AGAGGUAGUAGGUUGCAUAGUU |
| mmu-let-7e | UGAGGUAGGAGGUUGUAUAGUU |
| mmu-let-7f | UGAGGUAGUAGAUUGUAUAGUU |
| mmu-let-7g | UGAGGUAGUAGUUUGUACAGUU |
| mmu-let-7i | UGAGGUAGUAGUUUGUGCUGUU |
| mmu-miR-100 | AACCCGUAGAUCCGAACUUGUG |
| mmu-miR-100 | AACCCGUAGAUCCGAACUUGUG |
| mmu-miR-122 | UGGAGUGUGACAAUGGUGUUUG |
| mmu-miR-127 | UCGGAUCCGUCUGAGCUUGGCU |
| mmu-miR-128 | UCACAGUGAACCGGUCUCUUU |
| mmu-miR-129-5p | CUUUUUGCGGUCUGGGCUUGC |
| mmu-miR-134 | UGUGACUGGUUGACCAGAGGGG |
| mmu-miR-140 | CAGUGGUUUUACCCUAUGGUAG |
| mmu-miR-145 | GUCCAGUUUUCCCAGGAAUCCCU |
| mmu-miR-149 | UCUGGCUCCGUGUCUUCACUCCC |
| mmu-miR-18a | UAAGGUGCAUCUAGUGCAGAUAG |
| mmu-miR-18b | UAAGGUGCAUCUAGUGCUGUUAG |
| mmu-miR-193 | AACUGGCCUACAAAGUCCCAGU |
| mmu-miR-199a-5p | CCCAGUGUUCAGACUACCUGUUC |
| mmu-miR-199b | ACAGUAGUCUGCACAUUGGUUA |
| mmu-miR-216a | UAAUCUCAGCUGGCAACUGUGA |
| mmu-miR-216b | AAAUCUCUGCAGGCAAAUGUGA |
| mmu-miR-218 | UUGUGCUUGAUCUAACCAUGU |
| mmu-miR-26a | UUCAAGUAAUCCAGGAUAGGCU |
| mmu-miR-31 | AGGCAAGAUGCUGGCAUAGCUG |
| mmu-miR-345 | GCUGACCCCUAGUCCAGUGCUU |
| mmu-miR-34c | AGGCAGUGUAGUUAGCUGAUUGC |
| mmu-miR-362-5p | AAUCCUUGGAACCUAGGUGUGAAU |
| mmu-miR-378 | ACUGGACUUGGAGUCAGAAGG |
| (old mmu-miR-422b) | |
| mmu-miR-384-3p | AUUCCUAGAAAUUGUUCACAAU |
| mmu-miR-409-3p | GAAUGUUGCUCGGUGAACCCCU |
| mmu-miR-450a-5p | UUUUGCGAUGUGUUCCUAAUAU |
| mmu-miR-450b-5p | UUUUGCAGUAUGUUCCUGAAUA |
| mmu-miR-452 | UGUUUGCAGAGGAAACUGAGAC |
| mmu-miR-464 | UACCAAGUUUAUUCUGUGAGAUA |
| mmu-miR-465a-5p | UAUUUAGAAUGGCACUGAUGUGA |
| mmu-miR-465b-5p | UAUUUAGAAUGGUGCUGAUCUG |
| mmu-miR-465c-5p | UAUUUAGAAUGGCGCUGAUCUG |
| mmu-miR-468 | UAUGACUGAUGUGCGUGUGUCUG |
| mmu-miR-98 | UGAGGUAGUAAGUUGUAUUGUU |
| mmu-miR-99a | AACCCGUAGAUCCGAUCUUGUG |
| mmu-miR-99b | CACCCGUAGAACCGACCUUGCG |
| old mmu-miR-422b | CUGGACUUGGAGUCAGAAGGC |
| miR-106b | UAAAGUGCUGACAGUGCAGAU |
| miR-20b | CAAAGUGCUCAUAGUGCAGGUAG |
| miR-17 | CAAAGUGCUUACAGUGCAGGUAG |
| miR-291a | CAUCAAAGUGGAGGCCCUCUCU |
| miR-291b-5p | GAUCAAAGUGGAGGCCCUCUCC |
| miR-25 | CAUUGCACUUGUCUCGGUCUGA |
| miR-32 | UAUUGCACAUUACUAAGUUGCA |
| miR-92a-1 | UAUUGCACUUGUCCCGGCCUG |
| miR-92a-2 | UAUUGCACUCGUCCCGGCCUCC |
| miR-92b | UAUUGCACUCGUCCCGGCCUCC |
| miR-367 | AAUUGCACUUUAGCAAUGGUGA |
| miR-19b | UGUGCAAAUCCAUGCAAAACUGA |
| miR-290-5p | ACUCAAACUAUGGGGGCACUUU |
| miR-292 | ACUCAAACUGGGGGCUCUUUUG |
| miR-200c | UAAUACUGCCGGGUAAUGAUGGA |
| miR-20a | UAAAGUGCUUAUAGUGCAGGUAG |
| miR-291b-3p | AAAGUGCAUCCAUUUUGUUUGU |

Moreover, to enhance the efficiency of establishing induced pluripotent stem (iPS) cells, the following cytokines and/or small molecules, may further be introduced into somatic cells, in addition to miRNA and mRNA of the invention, to be reprogrammed: i.e., basic fibroblast growth factor (bFGF), stem cell factor (SCF), etc. (cytokines); and histone deacetylase inhibitors such as valpronic acid, DNA methylase inhibitors such as 5′-azacytidine, histone methyltransferase (G9a) inhibitors such as BIX01294 (BIX), (small molecules) (D. Huangfu et al., Nat. Biotechnol., 26, pp. 795-797, 2008; S. Kubicek et al., Mol. Cell, 25, pp. 473-481, 2007; Y. Shi et al., Cell Stem Cell, 3, 568-574, 2008, Yan Shi et al., Cell Stem Cell, 2, pp. 525-528, 2008. In addition, p53 inhibitors such as shRNA or siRNA for p53 and/or UTF1 may be introduced into somatic cells (Yang Zhao et al., Cell Stem Cell, 3, pp 475-479, 2008). Also, activation of the Wnt signal (Marson A. et al., Cell Stem Cell, 3, pp 132-135, 2008) or inhibition of signaling by mitogen-activated protein kinase or glycogen synthase kinase-3 (Silva J. et al., PoS Biology, 6, pp 2237-2247 2008) can serve as a means for increasing the efficiency of generating iPS cells.

Identification of IPS Cells

The invention provides for methods of determining if a cell is a pluripotent stem cell. These methods include but are not limited to teratoma assays; antibody staining for Oct4, NANOG, Rex-1, SSEA3, SSEA4, SSEA1 (mouse only), Tra-1-60, Tra-1-80; morphological observations; RT-PCR for pluripotency factors; methylation pattern comparisons to hES cells (bisulfate sequencing); spontaneous differentiation to all three germ layers (analyzed by RT-PCR or Ab staining); and pluritest analysis.

A cell can also be determined to be a pluripotent stem cell by analysis of the presence or absence of various markers specific to undifferentiated cells, for example, by RT-PCR. For example, some pluripotent cell markers include: Oct3/4; Nanog; alkaline phosphatase (AP); ABCG2; stage specific embryonic antigen-1 (SSEA-1); SSEA-3; SSEA-4; Tra-1-60; TRA-1-81; Tra-2-49/6E; ERas/ECAT5, E-cadherin; βIII-tubulin; .alpha.-smooth muscle actin (.alpha.-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); and T-cell lymphoma breakpoint 1 (Tcl1); DPPA3/Stella; DPPA4. Other markers can include Dnmt3L; Sox15; Stat3; Grb2; SV40 Large T Antigen; HPV16 E6; HPV16 E7, β-catenin, and Bmi1. Such cells can also be characterized by the down-regulation of markers characteristic of the differentiated cell from which the iPS cell is induced. For example, iPS cells derived from fibroblasts may be characterized by down-regulation of the fibroblast cell marker Thy1 and/or up-regulation of SSEA-3 and 4. It is understood that the present invention is not limited to those markers listed herein, and encompasses markers such as cell surface markers, antigens, and other gene products including ESTs, RNA (including microRNAs and antisense RNA), DNA (including genes and cDNAs), and portions thereof.

iPS cells may be further identified by semipermanent cell proliferation, pluripotency, or cell morphology (Takahashi, K. et al., Cell 131:861-872 (2007)). Briefly, regarding semipermanent proliferation, the ability of cells to expand exponentially is tested by culturing the cells over about 4-6 months. In the case of human iPS cells, because the population doubling time is known to be about 46.9.±.12.4 hr, 47.8.±.6.6 hr, or 43.2±11.5 hr for example, this value can be indicative of the ability of proliferation. Alternatively, high telomerase activity may be detected by the telomeric repeat amplification protocol (TRAP) because iPS cells normally have high telomerase activity.

Pluripotency can be confirmed by forming teratoma and identifying tissues or cells of three embryonic germ layers (i.e., ectoderm, mesoderm, and endoderm). Specifically, cells are injected intradermally in a nude mouse (where the cells are induced from murine somatic cells) or in the spermary of a SCID mouse (where the cells are induced from human somatic cells), followed by confirming the formation of a tumor then confirming that the tumor tissues are composed of tissues including neural rosettes (ectoderm), cartilage (mesoderm), cardiac myocyte (mesoderm), gut-like epithelium (endoderm), adipose (mesoderm), and the like.

Because human or mouse iPS cell colonies are known to have a morphology similar to that of human or mouse ES cell colonies, the morphology of iPS cells can be used as an indicator of pluripotency. In general, human iPS cells form flat colonies, while mouse iPS cells tend to form swollen colonies.

Kits of Pharmaceutical Systems

The present invention provides for kits for producing a pluripotent stem cell or pharmaceutical compositions comprising iPS cells of the invention. Kits according to this aspect of the invention comprise a carrier means, in combination with an mRNA and miRNA of the invention. In one embodiment, a kit of the invention further comprises one or more of a culture medium suitable for producing pluripotent cells of the invention, a medium suitable for growth and maintenance of pluripotent cell colonies of the invention, and a transfection reagent. The carrier means may comprise any one of a box, carton, tube or the like, having in dose confinement therein one or more container means, such as vials, tubes, ampules, bottles and the like.

If desired, the kit is provided together with instructions for using the kit to produce pluripotent stem cells. The instructions will generally include information about how to produce pluripotent stem cells.

Formulations comprising pluripotent stem cells or differentiated cells derived from pluripotent stem cells of the invention may be provided in combination with carrier means and may include instructions that generally include information about the use of the cells for treating a subject having a disease. In other embodiments, the instructions include at least one of the following: description of the therapeutic agent (iPS cells or cells derived therefrom); warnings; indications; counter-indications; animal study data; clinical study data; and/or references. The instructions may be printed directly on the container (when present), as a label applied to the container, or as a separate sheet, pamphlet, card, or folder supplied in or with the container. In certain embodiments, kits of the invention may also include B18R protein or any other component known in the art to suppress an immune response.

Animal Models

The iPS cells of the invention are also applicable to animals, and may also be used to facilitate biomedical research of disease in a variety of animal model systems.

Uses

The methods of the invention provide for production of pluripotent stem cells that can be used for clinical applications including disease treatment and prevention. In particular, the iPS cells of the invention, or differentiated progeny cells can be used for applications in the field of regenerative medicine. The cells of the invention also provide for methods of designing personalized treatments for subjects in need thereof.

The iPS cells of the invention and their differentiated progeny can also be used to identify compounds with a particular function, for example, treatment or prevention of disease, determine the activity of a compound of interest and or determine the toxicity of a compound of interest. Further, the present invention provides a stem cell therapy comprising transplanting somatic cells into a patient, wherein the somatic cells are obtained by inducing differentiation from induced pluripotent stem cells that are obtained according to the methods of the invention by using somatic cells isolated and collected from a patient.

In addition, the present invention provides a method for evaluation of physiological effect or toxicity of a compound, a drug, or a toxic agent, with use of various cells obtained by inducing differentiation from induced pluripotent stem cells that are obtained by the methods of the invention.

The application of induced pluripotent stem cells produced by the method of the present invention is not specifically limited, and these cells can be used for every examination/study to be performed with use of ES cells, and for any disease therapy which utilizes ES cells. For example, induced pluripotent stem cells obtained by the method of the present invention can be induced to produce desired differentiated cells or precursor cells (such as nerve cells, myocardial cells, blood cells and insulin-producing cells) or by treatment with retinoic acid, a growth factor such as EGF, glucocorticoid, activin A/BMP4 (bone morphogenetic protein 4), or VEGF (vascular endotherial growth factor), so that appropriate tissues can be formed. Stem cell therapies through autologous cell transplantation can be achieved by returning these differentiated cells or tissue obtained in the above manner, into the patient. However, the application of the induced pluripotent stem cells (iPS cells) of the present invention is not to be limited to the abovementioned specific aspects. The iPS cells have a capacity of germline transmission in vivo. Thus, when the iPS cells are introduced into the blastocyst from a non-human mammal, and then transplanted into the uterus of a surrogate mother of the same animal, a chimeric animal to which part of the genotypes of the iPS cell has been transmitted (WO 2007/069666) is produced. The iPS cells can also be used for modification of a gene, introduction (or knock-in) of a gene, or knock-out of a gene, thereby enabling clarification of the function of a gene, to create a non-human animal model with disease, or to produce a substance such as protein.

Methods of Treatment

The present invention provides for both prophylactic and therapeutic methods of treating a subject at risk of (or susceptible to) a disease or disorder treatable via administration of the pluripotent stem cells of the invention or differentiated progenitor cells.

In one aspect, the invention provides a method for preventing in a subject, a disease or disorder as described above by administering to the subject an iPS or differentiated progenitor cell of the invention. Subjects at risk for the disease can be identified by, for example, any or a combination of diagnostic or prognostic assays as described herein. Administration of a prophylactic agent can occur prior to the detection of, e.g., cancer in a subject, or the manifestation of symptoms characteristic of the disease or disorder, such that the disease or disorder is prevented or, alternatively, delayed in its progression.

Another aspect of the invention pertains to methods of treating subjects therapeutically, i.e., altering the onset of symptoms of the disease or disorder. These methods can be performed in vivo (e.g., by administering the pluripotent stem cells or differentiated progeny of the invention to a subject).

Therapeutic agents (e.g. pluripotent cells of the invention) can be tested in an appropriate animal model. For example, a pluripotent stem cell or differentiated progeny cell, as described herein can be used in an animal model to determine the efficacy, toxicity, or side effects of treatment with the cell. Alternatively, an agent (e.g., a pluripotent stem cell of the invention) can be used in an animal model to determine the mechanism of action of such an agent.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The present invention is described by reference to the following Examples, which are offered by way of illustration and are not intended to limit the invention in any manner. Standard techniques well known in the art or the techniques specifically described below were utilized.

### Example 1

### Transfection of eGFP mRNA Using the Stemfect™ RNA Transfection Kit

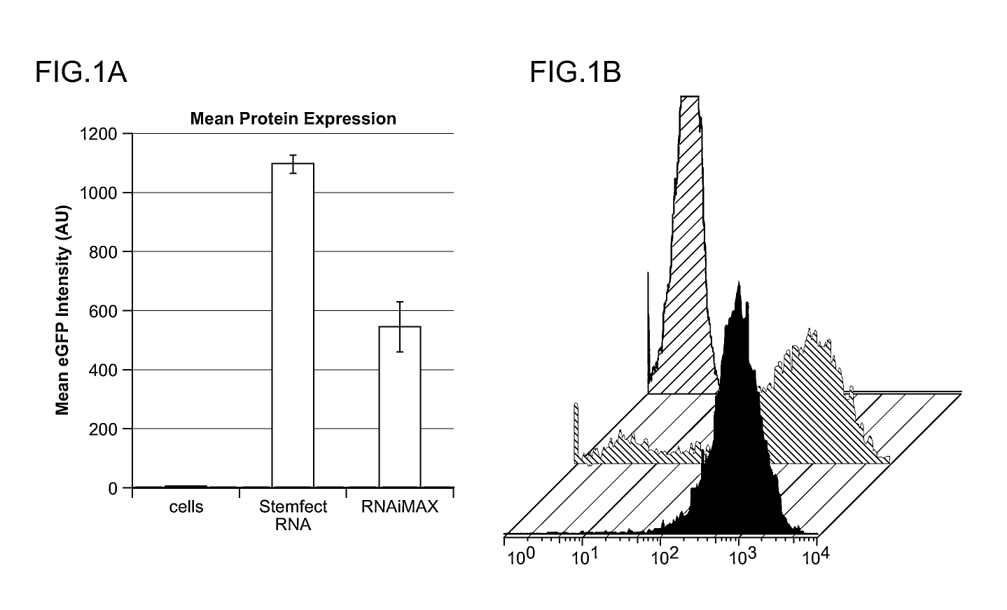

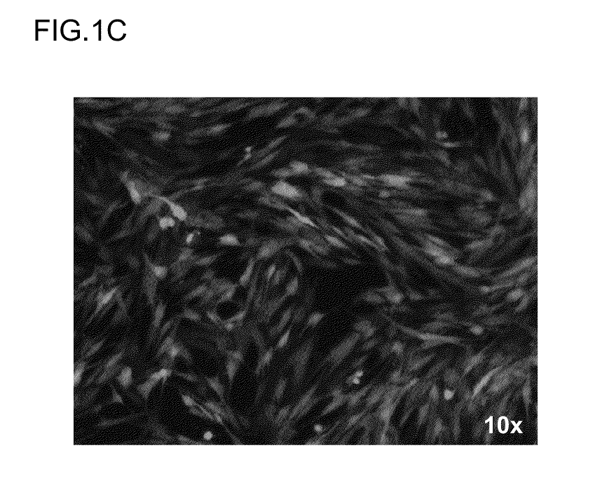

FIGS. 1A-C demonstrate the results of experiments wherein fibroblasts are transfected with eGFP mRNA using the Stemfect™ RNA Transfection Kit. BJ fibroblast cells (fibroblasts derived from human foreskin that are not mature) were seeded in a 24-well format and transfected with 250 ng of eGFP mRNA. The cells were cultured at 37° C. and 5% CO₂ and analyzed by flow cytometry at 18-24 hours post-transfection. FIG. 1A is a graph demonstrating the mean fluorescence intensity as determined by flow cytometry. Stemfect™ RNA Transfection Kit yielded 2-3 fold higher average protein expression than that observed using RNAiMAX™. FIG. 1B presents representative histograms comparing the transfection efficiency of Stemfect™ RNA Transfection Kit (purple) to RNAiMAX™ (green) alongside an untransfected cells control (red). Stemfect™ Transfection Kit led to >98% transfection efficiency of eGFP mRNA without any significant toxicity, while enabling a tighter distribution of protein expression.

FIG. 1C presents the results of an experiment wherein 75,000 BJ fibroblasts were seeded in 24-well format and transfected with 250 ng of eGFP mRNA using the Stemfect™ RNA Transfection Kit. Fluorescent image captured 18-24 hours post-transfection.

### Example 2

### Derivation of Integration-Free iPS Cells from Primary Patient Fibroblasts in a Feeder-Free Environment

iPS cells of the invention are generated from primary patient fibroblasts in a feeder-free environment.

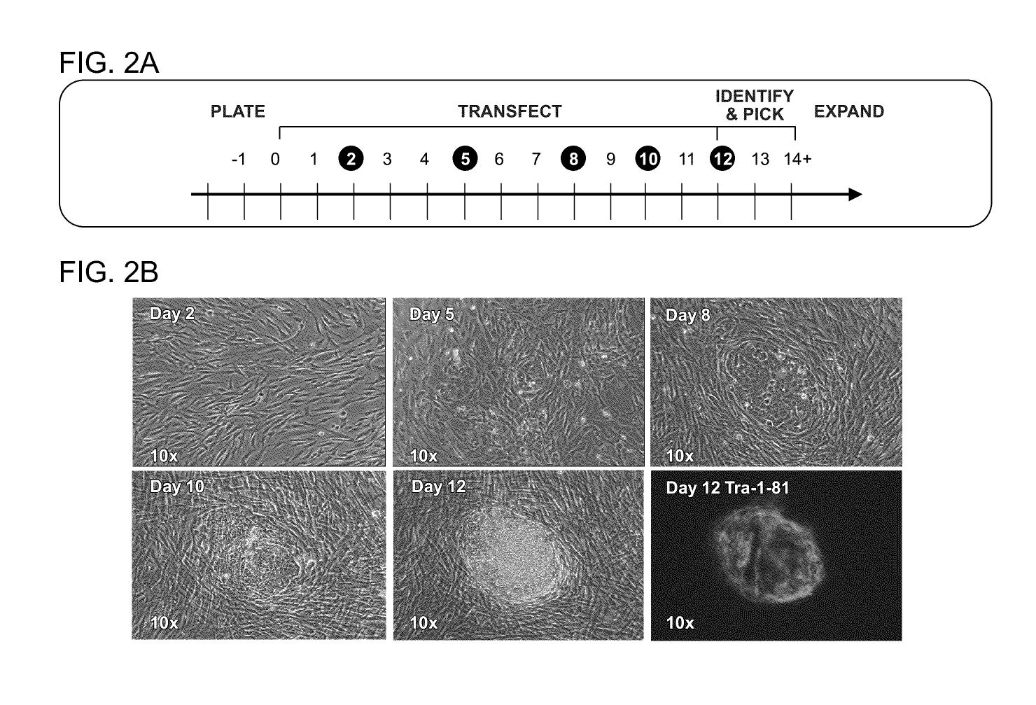

The Experimental timeline for production of iPS cells from primary patient fibroblasts in a feeder free environment is presented in FIG. 2A.

Experimental Timeline:

On day 1, 50,000 human fibroblasts were seeded in a single well of a 6-well plate, pre-coated with Matrigel™ and cultured overnight at 37° C., 5% CO₂, and 21% O₂. During days 0-12 target fibroblasts were transfected in medium previously conditioned with NuFFs (Human Newborn Foreskin Fibroblasts). The cells were transfected with miRNA and mRNA cocktail of the invention (for example, Cluster A or Cluster B) as follows:

Day 0-pluripotency miRNA cocktail;

Days 1-3-1.5 μg of mRNA cocktail (OSKML-Oct4, Sox2, Klf4, Myc and Lin28);

Day 4 □both mRNA and miRNA cocktails (sequentially added);

Days 5-12-1.5 μg of mRNA cocktail.

FIG. 2B presents the morphology Progression. 50,000 diseased patient dermal fibroblasts were seeded in one well of a 6-well plate and were then transfected as outlined above. Images were captured at defined time-points (purple dots in FIG. 2A). Day 2: The fibroblasts display typical compact morphologies in response to repeated transfection with mRNA. Day 5: Cells have initiated mesenchymal to epithelial transition and begin to assemble into small, loose clusters. Day 8: The rate of proliferation of cells within the dusters has increased as the edges of the colonies are emerging. Day 10: The duster of cells has expanded, and the edges of a burgeoning colony are more well defined. Day 12: TRA-1-81(+) iPS cell colonies with defined edges and tight cell clustering are present in the primary culture. TRA-1-81 is a surface marker for pluripotency.

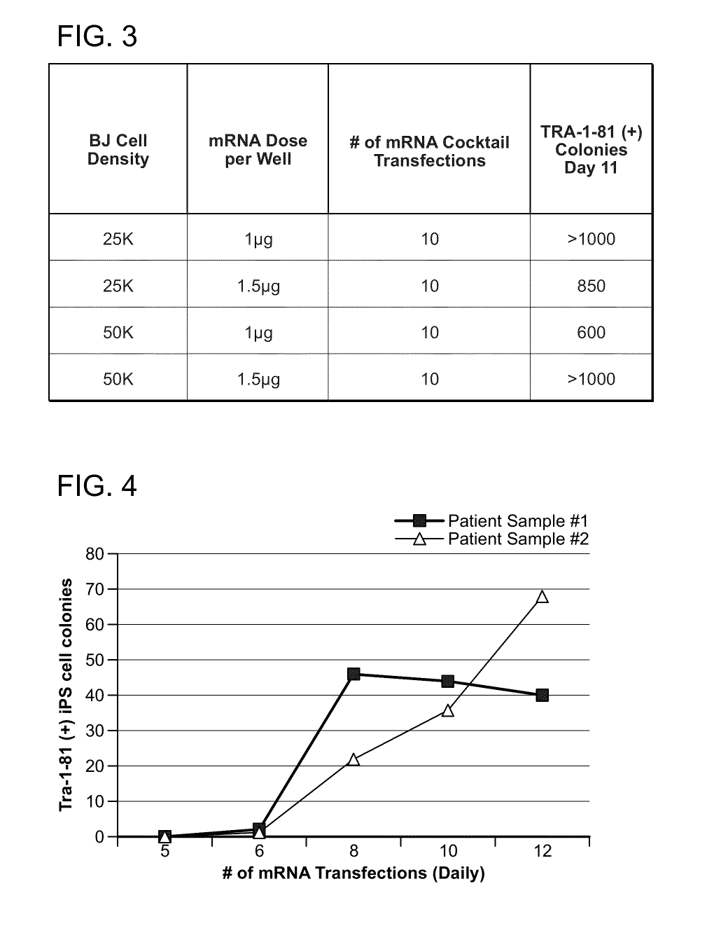

FIG. 3 presents the effect of target cell number and mRNA dose on iPS cell generation. Human fibroblasts were seeded at either 25,000 or 50,000 cells per well on a Matrigel™ coated 6-well plate and allowed to adhere overnight. The cells were transfected daily with either 1.0 or 1.5 μg mRNA (encoding at least one of Oct4, Sox2, Klf4, Myc and Lin28) in NuFF conditioned Medium containing 300 ng/ml B18R protein for 10 days. Cultures were incubated at 37° C., 5% CO₂, and 5% O₂. Wells were assessed for the number of TRA-1-81 positive colonies at Day 11.

Transfection of mRNA elicits an immune response from the cells that ultimately leads to apoptosis and death in the cell culture. This response is abrogated by using modified nucleotide or by using the B18R protein to block the immune response (see Angel and Yannik PLOS ONE, 2010)

### Example 3

### Number of Transfections Required for Generating iPS Cell Colonies

The number of transfections required for generating iPS cell colonies when transfecting with an mRNA cocktail only was determined. Two patient derived human dermal fibroblast cultures were each seeded at 50,000 cells in one well of a Matrigel™ coated 6-well plate and cultured overnight at 37° C., 5% CO₂, and 21% O₂. Cells were transfected daily with 1.5 μg of mRNA reprogramming cocktail in Pluriton™ Reprogramming Medium for the indicated number of days (see FIG. 4) and incubated overnight at 37° C., 5% CO₂, and 21% O₂. After completing the transfections, the media was changed daily until Day 12. Each well was then individually stained with Stemgent StainAlive™ (Stemgent) TRA-1-81 Antibody for iPS cell colony identification to assess reprogramming productivity at Day 12. Colonies emerged in wells receiving as few as 6 transfections. Maximal iPS cell colony productivity was observed when primary patient fibroblasts samples received 8 to 12 mRNA transfections

### Example 4

### iPS Cell Colony Formation in a Scaled-Down Format and Atmospheric Oxygen Tension

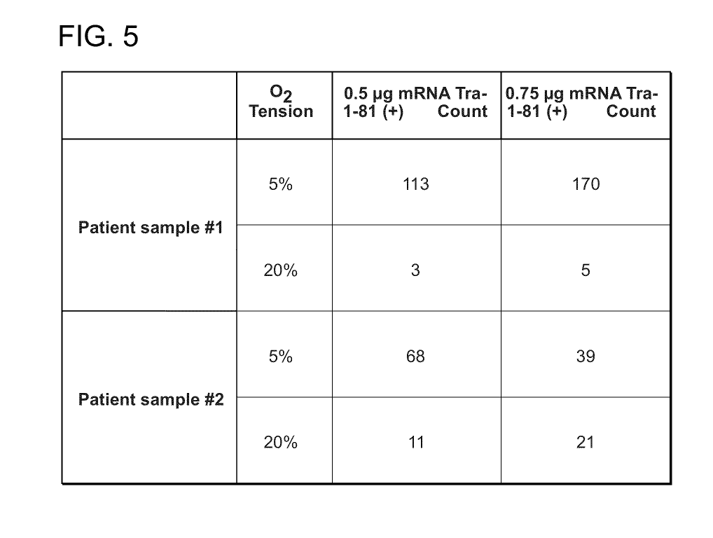

As demonstrated in FIG. 5, addition of miRNA supports iPS cell colony derivation in a scaled-down, 12-well format and atmospheric oxygen tension. Two patient-derived dermal fibroblast cultures were seeded at 25,000 cells per well of a Matrigel™ coated 12-well plate and cultured overnight at 37° C. and 5% CO₂. The cultures were then transfected with either 0.5 μg or 0.75 μg of mRNA cocktail Oct4, Sox2, Klf4, c-Myc, Lin28 and nGFP at a molar ration of 3:1:1:1:1:1 and miRNA cocktail, for example, Cluster A or Cluster B, under indicated O₂ tensions according to the timeline outlined in FIG. 2A. Wells at 5% O₂ were counted on Day 13, while wells at 21% O₂ were counted on Day 16 due to delayed emergence of iPS cell colonies. Each well was individually assayed with Stemgent StainAlive™ TRA-1-81 Antibody and counted to assess reprogramming productivity. Inclusion of miRNA cocktail allows iPS cell colony generation in a 12-well culture format in both reduced (5%) and atmospheric (21%) oxygen tensions.

### Example 5

### Continued Expansion and Maintenance of Pluripotency of Clonal mRNA iPS Cell Lines Under Feeder-Free Conditions

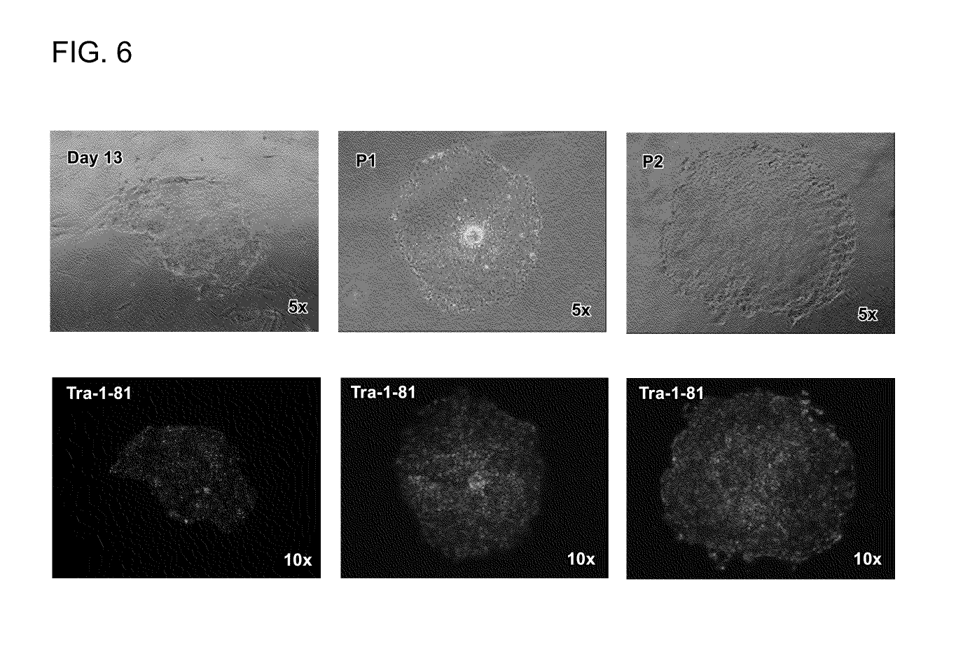

FIG. 6 demonstrates the continued expansion and maintenance of pluripotency of clonal mRNA iPS cell lines under feeder-free conditions. A primary mRNA iPS cell colony derived in Pluriton™ Reprogramming Medium on Matrigel™ was manually isolated at Day 13 and continued to express surface markers for pluripotency (TRA-1-81), as it was subsequently passaged in NutriStem™ XF/FF Culture Medium on Matrigel™, resulting in an integration-free, virus-free iPS cell line that has never been in contact with a feeder layer.

### Example 6

### The Use of miRNA to Enable Reprogramming in Refractory Lines

Using miRNA mimics in conjunction with mRNA, iPS cell colonies were generated from cell lines that are refractory to methods involving mRNA alone or miRNA alone. These target cells were primary patient fibroblasts seeded onto a feeder layer at 5,000 cells/well. Typically, reprogramming experiments require >100,000 cells/well in a 6-well format. According to the novel claimed methods, such high numbers of target cells are not required. In one embodiment, the novel methods provides for production of pluripotent stem cells from 1,000-10,000 cells/well in a 6-well format.

As indicated in the timeline presented below, cells were treated with miRNA (cluster A or B) at day 0 and prior to any mRNA transfection. They were then cotransfected with miRNA and mRNA, wherein the mRNA encodes at least one of Oct4, Sox2, Klf4, Myc and Lin28 at day 3. Every day through day 16, cells were treated with mRNA alone. In parallel, control cells were transfected with only mRNA for 16 straight days and compared to the miRNA+mRNA transfected cells grown under identical conditions. All cells were trypsinized and replated at specified cell densities on NuFFs at day 7. Cells treated with either miRNA cluster A (hsa-mir-302a, hsa-mir-302b, hsa-mir-302c, hsa-mir-302d and hsa-mir-367) or miRNA cluster B (hsa-mir-302a, hsa-mir-302b, hsa-mir-302c, hsa-mir-302d, hsa-mir-200c, hsa-mir-369-3p and hsa-mir-369-5p), in addition to mRNA encoding at least one of Oct4, Sox2, Klf4, Myc and Lin28, produced 1-2 colonies that stained positive for Tra-1-81, a pluripotent stem cell marker, while cells treated with mRNA alone did not yield any iPS cell colonies.
Feeder: Nuff 300 k/well
Target cells: primary patient fibroblasts-5 k/well, LN0005×3 well, LN0013×3 wells,
Media: LN-Media for first 5 days then Pluriton 2532
Protocol: mRNA alone or mRNA plus 2× miRNA transfection using RNAiMAX reagent, cells split at day 7, each well re-plated with 50 k and 20 k cells on Nuffs
   mRNA required: 1.2 ug/well (encoding at least one of Oct4, Sox2, Klf4, Myc and Lin28), R1 for LN-cells

| | | |
|---|---|---|
| LN0005, LN-medium, | LN0005, LN-medium | LN0005, LN-medium |
| mRNA | mRNA + miRNA | mRNA + miRNA |
| | cluster A | cluster B |
| LN0013, LN-medium, | LN0013, LN-medium, | LN0013, LN-medium, |
| mRNA | mRNA + miRNA | mRNA + miRNA |
| | cluster A | cluster B |

At day −2 NuFF were seeded onto 6-well plate at 300,000 cells/well. At day −1 LNH primary patient fibroblasts were seeded onto the feeder layer at 5,000 cells/well. At day 0 2 wells were transfected with mRNA and 4 wells were transfected with miRNA (2 wells with duster A and 2 wells with cluster B). At day 3 the 2 wells were transfected with mRNA and the 4 wells were co-transfected with mRNA+miRNA (2 wells with cluster A and 2 wells with duster B). On the remaining days all cells were transfected with mRNA through d18. At day 5 cells in the wells treated with mRNA+miRNA show more morphology changes than the wells with mRNA alone. At day 7 the cells are split and counted. The cell count per well is below:
LN005 mRNA control=5.35×10⁵
LN005 mRNA+miRNA cluster A=8.1×10⁵
LN005 mRNA+miRNA cluster B=8.1×10⁵
LN013 mRNA control=6.6×10⁵
LN013 mRNA+miRNA cluster A=1.12×10⁶
LN005 mRNA+miRNA cluster B=1.0×10⁶

The cells were re-plated at 100 k/well and 50 k/well for each condition

At day 14 iPS colonies appeared in the LN0013-mRNA+miRNA-B 100K/well sample. At days 20-d22 LiveStain Tra-1-81 was performed. 4 positive iPS, two iPS derived from LN0013 co-transfection with cluster A and B and two iPS derived from LN0005 co-transfection with duster A were detected and one iPS from each condition were expanded.

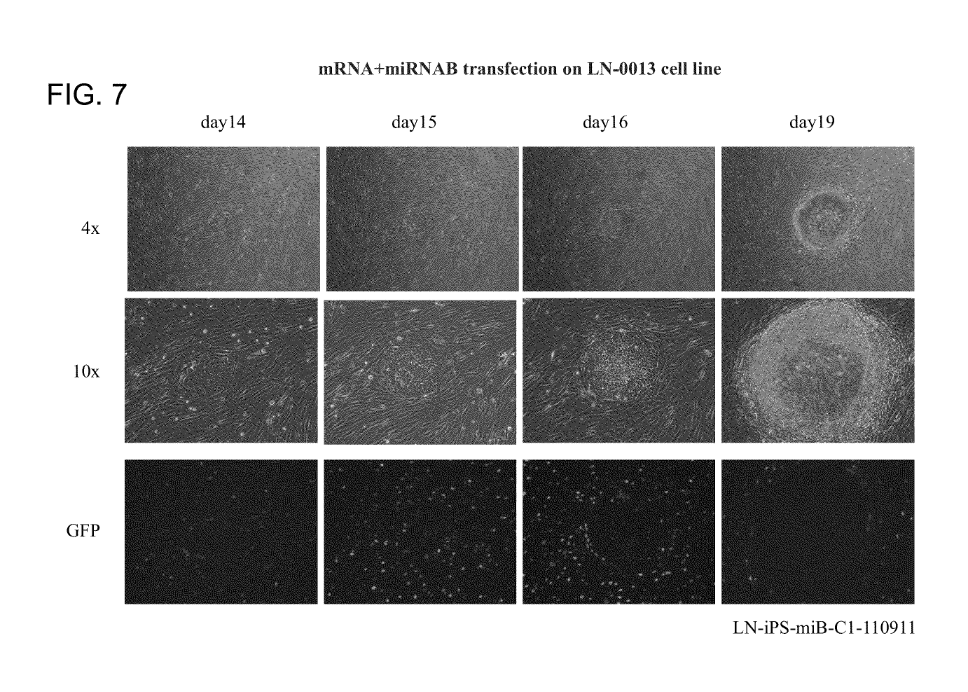

Results are presented in FIG. 7.

These data demonstrate that LN cell lines (primary patient fibroblasts) are difficult to be reprogrammed using mRNA alone (3× transfections were done). These data also demonstrate that miRNA, in combination with mRNA enhances cell proliferation and iPS reprogramming of patient primary fibroblast cells designated LN cells

### Example 7

### Comparison of miRNA to siRNA on Enhancement of iPS Cell Generation

These data present experiments wherein the ability of miRNA and siRNA to enhance iPS cell generation are compared.

Inhibition of p53 has been shown to increase cellular proliferation. The generation of iPS cells treated with mRNA encoding at least one of Oct4, Sox2, Klf4, Myc and Lin28 supplemented with p53 was compared to the generation of iPS cells treated with mRNA and miRNA cluster A or duster B (see details presented below). As in prior experiments, the target primary patient fibroblasts were seeded and grown on a NuFF layer for the duration of the experiment. The effect of splitting the culture on the output number of iPS cell colonies was also determined.

While previous attempts to reprogram certain primary patient fibroblasts with mRNA alone were unsuccessful, addition of miRNA cluster A or duster B increased the efficiency of mRNA reprogramming. In the no-split protocol starting with 5,000 cells/well 57 colonies under either cluster A or duster B conditions were produced. This efficiency of over 1% is higher than any efficiency typically observed in other reprogramming systems. In contrast, addition of siRNA targeting p53 had a minimal effect, yielding only 1 colony when starting with the same number of cells. mRNA, saRNA and miRNA Transfection on primary patient cells
Feeder: Nuff 300 k/well, 3002M lot#868
Target cells: For No split-primary patient fibroblasts: 5 k cells/well×3 wells (in one plate)
For split-primary patient fibroblasts: 10 k cells/well×3 wells (in other plate)
Media: Pluriton 2532 with supplement Lot#2567 B18R lot#1633
Protocol: Split 10 k wells after 6-7 transfections, re-plate each well (condition) at 50 and 100 k cells/well (6 wells from 3 of 10 k wells)
   mRNA required: 1.2 ug/well encoding at least one of Oct4, Sox2, Klf4, Myc and Lin28, R4
   mRNA transfection was performed every day for 4 h except day 0; miRNA and siRNA were added at day 0.
   miRNA for 4 wells/6-well plate was introduced into the cells by transfecting the cells for 4 h at day 0 and day 3 (day 3-miRNA was cotransfected with mRNA)
   miRNA—cluster A: miRNA 302A, 302B, 302C, 302D, 367
   miRNA—cluster B: miRNA 302A, 302B, 302C, 302D, 200C, 369-3p, 369-5p.

1 vial of miRNA powder (1.00D)+250 ul RNase free TE=20 uM stock was used.

Equal amount (ul) of each miRNA stock was mixed into the cocktail and aliquoted.

For transfections 3.5 ul of the cluster cocktail was adder per well/6-well plate/in 2 ml media
(20000 nM×3.5 ul=X ul×2000 ul, X=35 nM miRNA cluster A and cluster B, final concentration of miRNA in the well is 35 nM)
A tube: 7 ul miRNA A or B+117 ul Opti-M
B tube: 10.25 ul RNAiMAX+117 ul Opti-M
A and B were mixed and maintained at room temperature for 15 min. 120 ul of complex was added into each well

siRNA for 2 wells/6-well plate at day 0 and day 4 (day 4-cotransfected with mRNA):
p53 siRNA stock 20 pmol/ul
A tube: 1.5 ul siRNA+250 ul Opti-M
B tube: 5 ul RNAiMAX+250 ul Opti-M
A and B were mixed and maintained at room temperature for 15 min. 250 ul of complex was added into each well

6-Well Plate Format:

| | | |
|---|---|---|
| UTC 5K + miRNA | UTC 5K + miRNA | UTC 5K + p53 |
| cluster A | cluster B | siRNA |
| UTC 10K + miRNA | UTC 10K + miRNA | UTC 10K + p53 |
| cluster A | cluster B | siRNA |

At day −2: NuFF seeded onto 6-well plate at 300,000 cells/well

At day −1: primary patient fibroblasts seeded onto the feeder layer at 5,000 or 10,000 cells/well

At day 0: miRNA or siRNA transfections were performed. No mRNA transfections were performed at day 0.

At day 3:
2 wells were transfected with mRNA;
4 wells were transfected with mRNA+miRNA (2 wells with duster A and 2 wells with duster B);

At day 4:
The 4 wells were transfected with mRNA transfection (miRNA wells)
The 2 wells were transfected with mRNA+siRNA

Other days:
mRNA transfection was performed daily through d17

At days 4-5, early morphology changes were beginning to occur in the cells in the wells transfected with miRNA. Cells at a higher cell density were observed in cells treated with siRNA, although these cells exhibited fewer morphology changes.

Day 6: the culture was split and cells were plated (primary patient fibroblasts) at 10 k and the cells were counted. The cell count/well was as below
mRNA+miRNA A=9.1×10⁵
mRNA+miRNA B=8.77×10⁵
mRNA+siRNA A=8.3×10⁵

Cells were re-plated at 60 k, 40 k and 20 k/well for each condition

Day 12:

Certain small iPS appear in the 5 k-miRNA A and B wells

Many “loose” dusters were observed in the split wells-miRNA-A and B

Day 17: LiveStain Tra1-81 n No Split Wells and Colony Count

| UTC 5K + miRNA | UTC 5K + miRNA | UTC 5K + p53 |
|---|---|---|
| cluster A | cluster B | siRNA |
| 57 | 57 | 1 |

Day 18: LiveStain Tra1-81 in the Split Wells and Colony Count

| UTC 60K + miRNA | UTC 40K + miRNA | UTC 20K + miRNA |
|---|---|---|
| cluster A | cluster A | cluster A |
| 44 | 40 | 14 |
| UTC 60K + miRNA | UTC 40K + miRNA | UTC 20K + miRNA |
| cluster B | cluster B | cluster B |
| 68 | 58 | 21 |
| UTC 60K + P53 | UTC 40K + P53 | UTC 20K + P53 |
| siRNA | siRNA | siRNA |
| 4 | 7 | 3 |

These data demonstrate that UTC cells are not reprogrammed by the addition of mRNA alone. These data also demonstrate that UTC cells treated with miRNA are efficiently reprogrammed as compared with UTC cells treated with either mRNA alone or with mRNA and siRNA in combination.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or dearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise dearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

### REFERENCES

1. Warren, L., Manos, P. D., Ahfeldt, T., Loh, Y. H., Li, H., Lau, F., Ebina, W., Mandal, P. K., Smith, Z. D., Meissner, A., Daley, D. Q., Brack, A. S., Collins, J. J., Cowan, C., Schlaeger, T. M., Rossi, D. J. (2010) Highly efficient reprogramming to pluripotency and directed differentiation of human cells with synthetic modified mRNA. Cell Stem Cell, 7:618-30.
2. Angel, M., Yanik, M. F. (2010) Innate Immune Suppression Enables Frequent Transfection with RNA Encoding Reprogramming Proteins. PLoS One 5:e11756.
3. Yakubov, E., Rechavi, G., Rozenblatt, S., Givol, D. (2010) Reprogramming of Human Fibroblasts to Pluripotent Stem Cells using mRNA of Four Transcription Factors. Biochem Biophys Res Commun. 394:189.
4. Anokye-Danso, F., Snitow, M. and Morrisey, E. E. (2012) How microRNAs Facilitate Reprogramming to Pluripotency J. Cell Science 125, 1-9.
5. Subramanyam, D. Lamouille S., Judson, R. L., Liu, J. Y., Bucay, N., Derynck, R., Blelloch, R. (2011) Multiple Targets of miR-302 and miR-372 Promote Reprogramming of Human Fibroblasts to Induced Pluripotent Stem Cells Nature Biotechnology May; 29(5): 443-8.
6. U.S. 2013/0102768

We claim:

1. A method of producing a pluripotent stem cell comprising:
a. introducing at least one mRNA into a target cell;
b. introducing at least one miRNA into a target cell; and
c. culturing the target cell to produce a pluripotent stem cell.

2. The method of claim 1, wherein the step of introducing the at least one mRNA into the cell and/or the step of introducing the at least one miRNA into the target cell is repeated at least once.

3. The method of claim 1, wherein prior to step (a), at least one miRNA is introduced into the target cell.

4. The method of claim 1, wherein steps (a) and (b) are sequential.

5. The method of claim 1, wherein steps (a) and (b) occur on the same day.

6. The method of claim 1, wherein the stem cell is produced in less than 2 weeks from the initiation of step (a).

7. The method of claim 1, wherein the stem cell is produced in greater than 2 weeks from the initiation of step (a).

8. The method of claim 1, wherein the stem cell is produced in 2-3 weeks from the initiation of step (a).

9. The method of claim 1, wherein the stem cell expresses at least one of a surface marker selected from the group consisting of: SSEA3, SSEA4, Tra-1-81, Tra-1-60, Rex1, Oct4, Nanog and Sox2.

10. The method of claim 1, wherein the stem cells can divide in vitro for greater than one year; and/or divide in vitro for more than 30 passages; and/or stain positive by alkaline phosphatase or Hoechst Stain, and/or form a teratoma.

11. The method of claim 1, wherein the stem cell can form an embryoid body and express one or more endoderm markers selected from the group consisting of: AFP, FOXA2 and GATA4, and/or one or more mesoderm markers selected from the group consisting of: CD34, CDH2 (N-cadherin), COL2A1, GATA2, HAND1, PECAM1, RUNX1, RUNX2; and/or one or more ectoderm markers selected from the group consisting of: ALDH1A1, COL1A1, NCAM1, PAX6 and TUBB3 (Tuj1).

12. The method of claim 1, wherein, at least one stem cell is produced.

13. The method of claim 1, wherein one or both of the at least one miRNA and the at least one mRNA comprise a modified nucleotide.

14. The method of claim 1, wherein the at least one mRNA is not integrated into the genome of the stem cell.

15. The method of claim 1, wherein the mRNA and miRNA introduced into the target cell in steps (a) and (b) are not present in the stem cell.

16. The method of claim 1, wherein the culturing is performed in the absence of a feeder layer.

17. The method of claim 1, wherein the method is performed at ≦5% O₂.

18. The method of claim 1, wherein the method is performed at 5%-21% O₂.

19. The method of claim 1, wherein the method is performed at 21% O₂.

20. The method of claim 1, wherein the target cell is selected from the group consisting of: fibroblast, peripheral blood derived cells including but not limited to endothelial progenitor cell (L-EPCs)), cord blood derived cell types (CD34+), epithelial cells, and keratinocytes.

21. The method of claim 1, wherein the at least one mRNA encodes a reprogramming factor.

22. The method of claim 1, wherein the at least one mRNA encodes at least one of OCT4, SOX2, KLF4, c-MYC, LIN28, Nanog, Glis1, Sal4 and Esrbb1.

23. The method of claim 1, wherein the at least one miRNA comprises at least one miRNA that is 80% or more identical to an miRNA selected from the group consisting of hsa-miR-302a, hsa-miR-302b, hsa-miR-302c, hsa-miR302d, hsa-miR-367, hsa-miR-200c, hsa-miR-369-3p and hsa-miR-369-5p.

24. The method of claim 1, wherein the at least one miRNA comprises a combination of hsa-miR-302a, hsa-miR-302b, hsa-miR-302c, hsa-miR302d and hsa-miR367.

25. The method of claim 1, wherein the at least one miRNA comprises a combination of hsa-miR-302a, hsa-miR-302b, hsa-miR-302c, hsa-miR302d, hsa-miR-200c, hsa-miR-369-3p and hsa-miR-369-5p.

26. The method of claim 1, wherein the at least one miRNA comprises the combination of: hsa-miR-302a, hsa-miR-302b, hsa-miR-302c, hsa-miR-302d and hsa-miR-367; or hsa-miR-302a, hsa-miR-hsa-miR-302b, hsa-miR-302c, hsa-miR-302d, hsa-miR-200C, hsa-miR-369-3p, hsa-miR-369-5p; or the combination of hsa-miR-302a, hsa-miR-302b, hsa-miR-302c, hsa-miR-302d, hsa-miR-200C, hsa-miR-369-3p, hsa-miR-369-5p.

27. The method of claim 1, wherein the cell is a human cell.

28. A method of inducing pluripotency in a target cell comprising:
a. introducing at least one mRNA into the target cell;
b. introducing at least one miRNA into the target cell; and
c. culturing the target cell to produce a pluripotent cell.

29. An isolated pluripotent stem cell comprising at least one mRNA encoding a reprogramming factor in combination with at least one miRNA produced according to the method of claim 1.

30. The isolated pluripotent stem cell produced according to the method of claim 1, wherein the at least one mRNA is not integrated into the genome of the cell.

31. The isolated pluripotent stem cell produced according to the method of claim 1, wherein the mRNA and miRNA introduced into the target cell in steps (a) and (b) are not present in the stem cell.

32. The isolated pluripotent stem cell of claim 29, wherein the at least one miRNA comprises at least one miRNA that is 80% or more identical to an miRNA selected from the group consisting of hsa-miR-302a, hsa-miR-302b, hsa-miR-302c, hsa-miR302d, hsa-miR367, hsa-miR-200c, hsa-miR-369-3p and hsa-miR-369-5p.

33. The isolated pluripotent stem cell of claim 29, wherein the at least one mRNA encodes at least one of OCT4, SOX2, KLF4, c-MYC and LIN28.

34. A formulation comprising the isolated pluripotent stem cell of claim 29 or claim 47.

35. The formulation of claim 34, further comprising a compound that suppresses an immune response.

36. A kit for producing a pluripotent stem cell comprising at least one mRNA and at least one miRNA.

37. The kit of claim 36, further comprising culture media and a transfection reagent.

38. The kit of claim 36, further comprising a compound that suppresses an immune response.

39. A method of treating a subject with a disease comprising administering to the subject a cell produced by differentiation of the isolated pluripotent stem cell of claim 29 or the cell of claim 47.

40. A method of treating a subject with a disease comprising administering to the subject a cell produced by differentiation of the isolated pluripotent stem cell produced by the method of claim 1 or the cell of claim 47.

41. A method of identifying a compound for treatment of a disease comprising contacting a cell produced by differentiation of a stem cell produced by the method of claim 1 or the cell of claim 47 with a compound of interest.

42. A method of determining the activity of a compound for treating a disease comprising contacting a cell produced by differentiation of a stem cell produced by the method of claim 1 or the cell of claim 47 with a compound known to treat a disease.

43. A method of determining the toxicity of a compound for treating a disease comprising contacting a cell produced by differentiation of a stem cell produced by the method of claim 1 or the cell of claim 47 with a compound known to treat a disease.

44. The method of claim 40, wherein the cell is selected from the group consisting of: fibroblast, peripheral blood derived cells including but not limited to endothelial progenitor cell (L-EPCs)), cord blood derived cell types (CD34+), epithelial cells, and keratinocytes.

45. The method of claim 1, wherein the mRNA and/or the miRNA are not provided in a recombinant vector.

46. The method of claim 1, wherein the mRNA and/or the miRNA are not provided in a recombinant vector.

47. An isolated pluripotent stem cell comprising an mRNA in combination with an miRNA.

48. The isolated stem cell of claim 47, wherein neither of the mRNA and/or the miRNA is provided in a recombinant vector.

49. The isolated stem cell of claim 47, wherein neither of the mRNA and/or the miRNA is provided in a DNA vector or a viral vector.

50. The isolated stem cell of claim 47, wherein the stem cell expresses at least one of a surface marker selected from the group consisting of: SSEA3, SSEA4, Tra-1-81, Tra-1-60, Rex1, Oct4, Nanog and Sox2.

51. The isolated stem cell of claim 47, wherein the stem cells can divide in vitro for greater than one year; and/or divide in vitro for more than 30 passages; and/or stain positive by alkaline phosphatase or Hoechst Stain, and/or form a teratoma.

52. The isolated stem cell of claim 47, wherein the stem cell can form an embryoid body and express one or more endoderm markers selected from the group consisting of: AFP, FOXA2 and GATA4, and/or one or more mesoderm markers selected from the group consisting of: CD34, CDH2 (N-cadherin), COL2A1, GATA2, HAND1, PECAM1, RUNX1, RUNX2; and/or one or more ectoderm markers selected from the group consisting of: ALDH1A1, COL1A1, NCAM1, PAX6 and TUBB3 (Tuj1).

53. The isolated stem cell of claim 47, wherein the mRNA is not integrated into the genome of the stem cell.

54. The isolated stem cell of claim 47, wherein the mRNA and miRNA are exogenous.

55. The isolated stem cell of claim 47, wherein stem cell is derived from a cell selected from the group consisting of: fibroblast, peripheral blood derived cells including but not limited to endothelial progenitor cell (L-EPCs)), cord blood derived cell types (CD34+), epithelial cells, and keratinocytes.

56. The isolated stem cell of claim 47, wherein the mRNA encodes a reprogramming factor.

57. The isolated stem cell of claim 47, wherein the mRNA encodes at least one of OCT4, SOX2, KLF4, c-MYC, LIN28, Nanog, Glis1, Sal4 and Esrbb1.

58. The isolated stem cell of claim 47, wherein the miRNA comprises at least one miRNA that is 80% or more identical to an miRNA selected from the group consisting of hsa-miR-302a, hsa-miR-302b, hsa-miR-302c, hsa-miR302d, hsa-miR-367, hsa-miR-200c, hsa-miR-369-3p and hsa-miR-369-5p.

59. The isolated stem cell of claim 47, wherein the miRNA comprises a combination of hsa-miR-302a, hsa-miR-302b, hsa-miR-302c, hsa-miR302d and hsa-miR367.

60. The isolated stem cell of claim 47, wherein the miRNA comprises a combination of hsa-miR-302a, hsa-miR-302b, hsa-miR-302c, hsa-miR302d, hsa-miR-200c, hsa-miR-369-3p and hsa-miR-369-5p.

61. The isolated stem cell of claim 47, wherein the miRNA comprises the combination of: hsa-miR-302a, hsa-miR-302b, hsa-miR-302c, hsa-miR-302d and hsa-miR-367; or hsa-miR-302a, hsa-miR-hsa-miR-302b, hsa-miR-302c, hsa-miR-302d, hsa-miR-200C, hsa-miR-369-3p, hsa-miR-369-5p; or the combination of hsa-miR-302a, hsa-miR-302b, hsa-miR-302c, hsa-miR-302d, hsa-miR-200C, hsa-miR-369-3p, hsa-miR-369-5p.

62. The isolated stem cell of claim 47, wherein the cell is a human cell.

63. The isolated stem cell of any one of claim 29 or 47, wherein the mRNA and/or the miRNA are not provided in a recombinant vector.

64. The isolated stem cell of any one of claim 29 or 47, wherein the mRNA and/or the miRNA are not provided in a DNA vector or a viral vector.

65. The method of claim 1, wherein the mRNA and/or the miRNA is not provided in a recombinant vector.

66. The method of claim 1, wherein the mRNA and/or the miRNA is not provided in a DNA vector or a viral vector.

67. The method of claim 1, wherein step (b) precedes step a.

68. The method of claim 67, wherein step (b) is repeated at least once.

69. The method of claim 68, wherein the first occurrence of step (b) occurs from day 0 to day 1 and wherein step (b) is repeated at a time point selected from: day 1, until three weeks from the initiation of step (a).

70. The method of claim 1, wherein step (b) is repeated at least once and wherein the second occurrence of step (b) occurs on the same day as step (a).

71. The method of claim 1, wherein step (b) occurs from day 0 to day 1, step (b) occurs from day 4 through day 5 and step (a) occurs from day 1 through day 12.

72. The method of claim 1, wherein said miRNA comprises at least two miRNAs presented in Tables 1-6.

73. The method of claim 71, wherein said mRNA encodes at least one of OCT4, SOX2, KLF4, c-MYC, LIN28, Nanog, Glis1, Sal4 and Esrbb1.
